(19) United States
(12) Patent Application Publication
(10) Pub. No.: US 2020/0055934 A1
(43) Pub. Date: Feb. 20, 2020

(54) THERAPEUTIC AGENTS AND USE THEREOF

(71) Applicant: Swansea University, Swansea (GB)

(72) Inventors: Robert Conlan, Swansea (GB); Deyarina Gonzalez, Swansea (GB)

(73) Assignee: Swansea University, Swansea (GB)

(21) Appl. No.: 16/541,107

(22) Filed: Aug. 14, 2019

Related U.S. Application Data
(63) Continuation-in-part of application No. 15/519,826, filed on Apr. 17, 2017, now Pat. No. 10,406,124.

(30) Foreign Application Priority Data
Oct. 22, 2014 (GB) 1418809.8

(51) Int. Cl. C07K 16/28; A61K 47/65

(52) U.S. Cl. CPC C07K 16/2803; C07K 2317/21; A61K 47/65

(57) ABSTRACT

A therapeutic agent comprising a cell binding agent which binds the Receptor for Advanced Glycation Endproducts (RAGE) linked to an anti-cancer drug, for use in the treatment of gynaecological cancer, endometriosis or polycystic ovary syndrome. Novel cell binding agents, pharmaceutical compositions and methods are also described.

### CROSS REFERENCE TO RELATED APPLICATIONS

This is a continuation-in-part of application Ser. No. 15/519,826, filed Apr. 17, 2017, which is the national stage of International Application No. PCT/GB2015/05316, filed Oct. 21, 2015, which claims the benefit of Application No. GB 1418809.8, filed Oct. 22, 2014, all of which are incorporated herein by reference.

### FIELD

The present application relates to therapeutic agents, in particular antibody-drug conjugates, useful in the treatment of proliferative disease, in particular gynaecological cancers or polycystic ovary syndrome, in which the receptor for advanced glycation end products (RAGE) protein exhibits altered expression compared to physiologically normal tissues. Certain of the agents are novel and form a further aspect of the invention, as well as pharmaceutical compositions comprising the agents, methods for preparing them and their use in therapy.

### BACKGROUND OF THE INVENTION

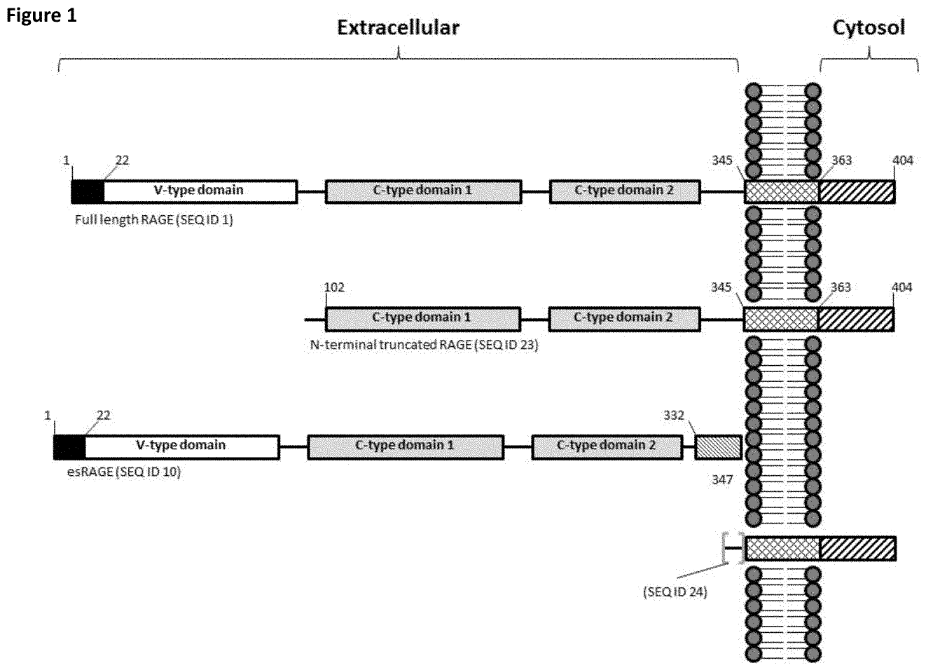

The receptor for advanced glycation end products (RAGE) is a member of the immunoglobulin superfamily of cell surface molecules,¹ located on chromosome 6p21.3 at the major histocompatibility complex class III region.² Full length RAGE is 404 amino acids in length, comprising an extracellular domain, a single hydrophobic transmembrane domain and a short cytosolic tail. Ligand binding properties are provided by the extracellular domain, which can be divided into three functional regions; the V domain, C1 and C2 domains (FIG. 1 hereinafter).³ An increasing number of ligands are known to bind RAGE including, advanced glycation end products (the receptors namesake), high-mobility group protein 1, and members of the S100 protein family.⁴⁻⁷ Central to its role in an inflammatory responses, is the internalisation of RAGE following ligand binding, which is a key component of RAGE-mediated signal transduction.⁸ Tissue distribution of RAGE under physiological conditions is limited, and with the exception of the lungs, expression is low.⁹

The up-regulation of RAGE expression is associated with a wide range of diseases, in particular in a range of inflammatory diseases such as diabetes and Alzheimer's disease.^{4,14} There is also evidence linking RAGE to cancer progression in mice and humans¹⁰⁻¹³.

Following the limited success of therapies which use monoclonal antibodies in the treatment of cancer, there has been some considerable interest in drug-antibody conjugates. The approach here is to attach to the antibodies, small molecule drugs, such as cytotoxins or other anti-cancer agents. The antibody acts as a targeting agent, carrying the drug directly to the tumour cell, and thus permitting discrimination between cancer cells and normal tissue.

However, initial work has shown that the selection of appropriate targets is critical for effective therapies to be developed.

Humanised anti-RAGE antibodies and therapeutic agents comprising them are described for example in WO2010/019656. It is suggested that they may be useful in a wide range of diseases in which RAGE is implicated.

The applicants have found that RAGE is upregulated in a number of specific cancers, including in particular gynaecological cancers such as endometrial or ovarian cancer. Furthermore, they have found that this receptor can be effectively targeted by antibodies in complex with cytotoxic drugs, thereby producing useful anti-cancer effects.

### SUMMARY OF THE INVENTION

According to the present invention there is provided a therapeutic agent comprising a cell binding agent which binds the receptor for advanced glycation end products (RAGE) linked to an anti-cancer drug, for use in the treatment of a proliferative disease selected from gynaecological cancer, endometriosis and polycystic ovary syndrome.

The cell binding agent is suitably one of, but without limitation to, an antibody or a binding fragment thereof, such as a Fab, Fab′, F(ab)2, F(ab′)2 and FV, VH and VK fragments; a peptide; an aptamer, a nanobody or other non-antibody affinity reagent. Antibodies may be monoclonal or polyclonal but in particular are monoclonal antibodies. Whilst the antibody may be from any source (murine, rabbit etc.), for human therapeutic use, they suitably comprise a human antibody or an antibody which has been partly or fully humanised.

The sequence of human RAGE is known, as well as a further twenty two variants including soluble RAGE (sRAGE). These are shown herein as SEQ ID NO 1 through SEQ ID NO 23, with full RAGE being SEQ ID NO 1 and sRAGE being SEQ ID No 2. The cell binding agent therefore is required to bind to an epitopic region of SEQ ID NO 1 or SEQ ID NO 2 or SEQ ID 3 or SEQ ID NO 4 or SEQ ID NO 5 or SEQ ID NO 6 or SEQ ID NO 7 or SEQ ID NO 8 or SEQ ID NO 9 or SEQ ID NO 10 or SEQ ID NO 11 or SEQ ID NO 12 or SEQ ID NO 13 or SEQ ID NO 14 or SEQ ID NO 15 or SEQ ID NO 16 or SEQ ID NO 17 or SEQ ID NO 18 or SEQ ID NO 19 or SEQ ID NO 20 or SEQ ID NO 21 or SEQ ID NO 22 or SEQ ID NO 23.

However, it is also known that RAGE is subject to protein ectodomain shedding.¹⁵ In a particular embodiment of the invention, the cell binding agent of the complex of the invention binds a region of the ectodomain of RAGE which remains after any such shedding occurs. For example amino acids 317 to 344 of SEQ ID NO 1, herein denoted as SEQ ID NO 24. In this way, the activity of the agent may be maximised since it might be expected to continue to act, even after shedding. In particular therefore, the therapeutic agent of the invention comprises a cell binding agent which binds a residual extracellular fragment of RAGE remaining after shedding of the ectodomain. In a particular embodiment therefore, the cell binding agent binds to an epitopic region of SEQ ID NO 24.

In another embodiment, the therapeutic agent of the invention comprises a cell binding agent which binds a V-type domain of the RAGE, where the V-type domain is found at amino acids 23 to 116 of SEQ ID NO 1. In yet another embodiment, the therapeutic agent binds a domain of RAGE for which MAB11451 is specific.

In a particular embodiment, the anti-cancer molecule used in the therapeutic is a cytotoxin, such as a small molecule cytotoxin, a hormone, a cytokine/chemokine or other cell signalling molecule, or a nucleic acid and shall hereinafter be referred to as an ‘anti-cancer drug.’

In particular, the anti-cancer drug is a cytotoxin that inhibits or prevents the function of cells and/or causes destruction of cells. Examples of cytotoxins include, but are not limited to, radioactive isotopes, chemotherapeutic agents, and toxins such as small molecule toxins or enzymatically active toxins of bacterial, fungal, plant or animal origin, including synthetic analogues and derivatives thereof. The cytotoxic agent may be selected from the group consisting of an auristatin, a DNA minor groove binding agent, a DNA minor groove alkylating agent, an enediyne, a lexitropsin, a duocarmycin, a taxane, a puromycin, a dolastatin, a maytansinoid and a vinca alkaloid or a combination of two or more thereof.

Other suitable anti-cancer drugs include topoisomerase inhibitors, alkylating agents (eg. nitrogen mustards; ethylenimes; alkylsulfonates; triazenes; piperazines; and nitrosureas), an antimetabolite (eg mercaptopurine, thioguanine, 5-fluorouracil), a mitotic disrupter (eg. plant alkaloids-such as vincristine and/or microtubule antagonists-such as paclitaxel), a DNA intercalating agent (eg carboplatin and/or cisplatin), a DNA synthesis inhibitor, a DNA-RNA transcription regulator, an enzyme inhibitor, a gene regulator, a hormone response modifier, a hypoxia-selective cytotoxin (eg. tirapazamine), an epidermal growth factor inhibitor, an anti-vascular agent (eg. xanthenone 5,6-dimethylxanthenone-4-acetic acid), a radiation-activated prodrug (eg. nitroarylmethyl quaternary (NMQ) salts) or a bioreductive drug or a combination of two or more thereof.

Non-limiting examples of chemotherapeutic agents include Auristatin, Erlotinib (TARCEVA®), Bortezomib (VELCADE®), Fulvestrant (FASLODEX®), Sutent (SU11248), Letrozole (FEMARA®), Imatinib mesylate (GLEEVEC®), PTK787/ZK 222584, Oxaliplatin (Eloxatin®), 5-FU (5-fluorouracil), Leucovorin, Rapamycin (Sirolimus, RAPAMUNE®), Lapatinib (GSK572016), Lonafarnib (SCH 66336), Sorafenib (BAY43-9006), and Gefitinib (IRESSA®), AG1478, AG1571 (SU 5271; Sugen) or combination of these.

The chemotherapeutic agent may be an alkylating agent-such as thiotepa, CYTOXAN® and/or cyclosphosphamide; an alkyl sulfonate-such as busulfan, improsulfan and/or piposulfan; an aziridine-such as benzodopa, carboquone, meturedopa and/or uredopa; ethylenimines and/or methylamelamines-such as altretamine, triethylenemelamine, triethylenepbosphoramide, triethylenethiophosphoramide and/or trimethylomelamine; acetogenin-such as bullatacin and/or bullatacinone; camptothecin; bryostatin; callystatin; cryptophycins; dolastatin; duocarmycin; eleutherobin; pancratistatin; sarcodictyin; spongistatin; nitrogen mustards-such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide and/or uracil mustard; nitrosureas-such as carmustine, chlorozotocin, fotemustine, lomustine, nimustine, and/or ranimnustine; dynemicin; bisphosphonates-such as clodronate; an esperamicin; a neocarzinostatin chromophore; aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN®. doxorubicin-such as morpholino-doxorubicin, cyanomorpholino-doxorubicin, 2-pyrrolino-doxorubicin and/or deoxydoxorubicin, epirubicin, esorubicin, idarubicin, marcellomycin, mitomycins-such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites-such as methotrexate and 5-fluorouracil (5-FU); folic acid analogues-such as denopterin, methotrexate, pteropterin, trimetrexate; purine analogues-such as fludarabine, 6-mercaptopurine, thiamiprine, thioguanine; pyrimidine analogues-such as ancitabine, azacitidine, 6-azauridine, carmofur, cytarabine, dideoxyuridine, doxifluridine, enocitabine, floxuridine; androgens-such as calusterone, dromostanolone propionate, epitiostanol, mepitiostane, testolactone; anti-adrenals-such as aminoglutethimide, mitotane, trilostane; folic acid replenisher-such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; macrocyclic depsipeptides such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofiran; spirogermanium; tenuazonic acid; triaziquone; 2,2′,2″-trichlorotriethylamine; trichothecenes-such as verracurin A, roridin A and/or anguidine; urethan; vindesine; dacarbazine; mannomustine; mitobronitol; mitolactol; pipobroman; gacytosine; arabinoside; cyclophosphamide; thiotepa; taxoids-such as TAXOL®. paclitaxel, abraxane, and/or TAXOTERE®, doxetaxel; chloranbucil; GEMZAR®. gemcitabine; 6-thioguanine; mercaptopurine; methotrexate; platinum analogues-such as cisplatin and carboplatin; vinblastine; platinum; etoposide; ifosfamide; mitoxantrone; vincristine; NAVELBINE®, vinorelbine; novantrone; teniposide; edatrexate; daunomycin; aminopterin; xeloda; ibandronate; topoisomerase inhibitor RFS 2000; difluoromethylomithine (DMFO); retinoids-such as retinoic acid; capecitabine; and pharmaceutically acceptable salts, acids, derivatives or combinations of these.

Examples of tubulin disrupters include taxanes-such as paclitaxel and docetaxel, vinca alkaloids, discodermolide, epothilones A and B, desoxyepothilone, cryptophycins, curacin A, combretastatin A-4-phosphate, BMS 247550, BMS 184476, BMS 188791; LEP, RPR 109881A, EPO 906, TXD 258, ZD 6126, vinflunine, LU 103793, dolastatin 10, E7010, T138067 and T900607, colchicine, phenstatin, chalcones, indanocine, T138067, oncocidin, vincristine, vinblastine, vinorelbine, vinflunine, halichondrin B, isohomohalichondrin B, ER-86526, pironetin, spongistatin 1, spiket P, cryptophycin 1, LU103793 (cematodin or cemadotin), rhizoxin, sarcodictyin, eleutherobin, laulilamide, VP-16 and D-24851 and pharmaceutically acceptable salts, acids, derivatives or combinations of these.

Examples of DNA intercalators include acridines, actinomycins, anthracyclines, benzothiopyranoindazoles, pixantrone, crisnatol, brostallicin, CI-958, doxorubicin (adriamycin), actinomycin D, daunorubicin (daunomycin), bleomycin, idarubicin, mitoxantrone, cyclophosphamide, melphalan, mitomycin C, bizelesin, etoposide, mitoxantrone, SN-38, carboplatin, cis-platin, actinomycin D, amsacrine, DACA, pyrazoloacridine, irinotecan and topotecan and pharmaceutically acceptable salts, acids, derivatives or combinations of these.

The drug may be an anti-hormonal agent that acts to regulate or inhibit hormone action on tumours-such as anti-estrogens and selective estrogen receptor modulators, including, but not limited to, tamoxifen, raloxifene, droloxifene, 4-hydroxytamoxifen, trioxifene, keoxifene, LY117018, onapristone, and/or fareston toremifene and pharmaceutically acceptable salts, acids, derivatives or combinations of two or more of any of the above. The drug may be an aromatase inhibitor that inhibits the enzyme aromatase, which regulates estrogen production in the adrenal glands-such as, for example, 4(5)-imidazoles, aminoglutethimide, megestrol acetate, AROMASIN®. exemestane, formestanie, fadrozole, RIVISOR®. vorozole, FEMARA®. letrozole, and ARIMIDEX® and/or anastrozole and pharmaceutically acceptable salts, acids, derivatives or combinations of two or more of any of the above.

Other anti-cancer drugs include anti-androgens-such as flutamide, nilutamide, bicalutamide, leuprolide, goserelin and/or troxacitabine and pharmaceutically acceptable salts, acids, derivatives or combinations of any of these. Alternatively, the anti-cancer drug may be a protein kinase inhibitor (including a cyclin dependent kinase inhibitor), a lipid kinase inhibitor or an anti-angiogenic agent.

In a particular embodiment, the drug is a dolastatin. Dolastatins are antiproliferative agents, inhibiting the growth and reproduction of target cells and inducing apoptosis in a variety of malignant cell types. Two natural dolastatins, dolastatin 10 and dolastatin 15, have been selected for drug development based on their superior antiproliferative bioactivity. The pursuit of synthetic dolastatin analogues has led to the development of LU103793 (cematodin or cemadotin), a dolastatin 15 analogue. ILX-651 is an orally active third generation synthetic dolastatin 15 analogue. In one embodiment, the dolastatin is of the auristatin class. As used herein, the term dolastatin encompasses naturally occurring auristatins and non-naturally occurring derivatives, for example monomethyl auristatin E (MMAE)((S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide) or monomethyl auristatin F (MMAF)((S)-2-((2R,3R)-3-((S)-1-((3R,4S,5S)-4-((S)—N,3-dimethyl-2-((S)-3-methyl-2-(methylamino)butanamido)butanamido)-3-methoxy-5-methylheptanoyl)pyrrolidin-2-yl)-3-methoxy-2-methylpropanamido)-3-phenylpropanoic acid).

Alternatively, the anti-cancer drug may comprise a nucleic acid such as an RNA molecule or nanomolecule which targets an oncogene gene, in particular an RNA molecule such as a small interfering RNA (siRNA), a short hairpin RNA (shRNA), a microRNA (miRNA), or a short activating RNA (saRNA) which are designed to silence or activate genes, and in particular oncogenes. A wide variety of such RNAs are known, and the therapeutic potential of these molecules has been extensively reviewed.^{16,17}

The therapeutic agent of the invention comprises a cell binding agent linked to an anti-cancer drug as defined above. The means by which these two entities are linked together will depend upon factors such as the nature of the cell binding agent and the specific nature of the drug. In a particular embodiment, the cell binding agent is linked to the anti-cancer drug by way of a chemical linking group. The chemical linking group is suitably covalently bonded to both the cell binding agent and the anti-cancer drug. It is suitably such that it breaks down in the cell in-vivo to release the anti-cancer drug in a potent form.

Examples of suitable linkers may be chemically-labile, such as acid-cleavable hydrazine linkers or disulphide bonds; enzymatically-labile, such as peptide linkers or carbohydrate moieties; or non-cleavable linkers, such as thioether linkers or amides, as are known in the art.¹⁸

Generally, a chemical entity comprising the linker group is reacted with the cell binding agent under conditions in which the linker group becomes attached to the cell binding agent, either by conjugation or by covalent bonding.

In a particular embodiment, the chemical entity comprising the linker group is a maleimidocaproyl-valine-citrullin-p-aminobenzyloxycarbonyl linker. This linker is ‘self-immolative’ in the sense that it breaks down in vivo in a cell to release the anti-cancer drug. The linker exhibits high plasma stability and a protease cleavage site. Enzymatic cleavage leads to 1, 6-elimination of the 4-aminobenzyl group, releasing the anti-cancer drug.^{18,19}

The relative amount of drug: cell binding agent may be varied and will depend upon the relative amount of linker applied to the cell binding agent. It should be sufficient to provide a useful therapeutic ratio for the agent, but the loading should not be so high that the structure of the cell binding agent and in particular its ability to enter the cell via the RAGE receptor is compromised. The amounts will therefore vary depending upon the particular cell binding agent and the particular anti-cancer drug used. However, typically the ratio of drug:cell binding agent molecules in the therapeutic agent is in the range of from 1:1 to 1:8, for example from 1:1.5 to 1:3.5.

The therapeutic agents described above are useful in the treatment of gynaecological proliferative disease. In particular, the applicants have found that the cell binding agent will bind to the RAGE receptor of a cell, in particular a gynaecological tumour cell, and become internalised within the cell. At this stage, any chemical linkers may be cleaved or the cell binding agent metabolised allowing the anti-cancer drug or an active metabolite to produce the desired effect. The applicants have found that therapeutic agents of this type are effective against human gynaecological cancer cells as illustrated hereinafter.

The therapeutic agent of the invention is used in the treatment of gynaecological proliferative conditions in which RAGE is overexpressed. The applicants have found that such proliferative conditions include gynaecological cancers such as endometrial or ovarian cancer, as well as endometriosis and polycystic Ovary Syndrome. For example, the agent is used to treat gynaecological cancers as described above, or polycystic Ovary Syndrome

For use in these therapies, the therapeutic agents of the invention are suitably administered in the form of a pharmaceutical composition.

Thus a further aspect of the invention provides a pharmaceutical composition comprising a therapeutic agent as described above in combination with a pharmaceutically acceptable carrier.

Suitable pharmaceutical compositions will be in either solid or liquid form. They may be adapted for administration by any convenient peripheral route, such as parenteral, oral, vaginal or topical administration or for administration by inhalation or insufflation. The pharmaceutical acceptable carrier may include diluents or excipients which are physiologically tolerable and compatible with the active ingredient. These include those described for example in Remington's Pharmaceutical Sciences.²⁰

Parenteral compositions are prepared for injection, for example subcutaneous, intramuscular, intradermal, and intravenous or via needle-free injection systems. Also, they may be administered by intraperitoneal injection. They may be liquid solutions or suspensions, or they may be in the form of a solid that is suitable for solution in, or suspension in, liquid prior to injection. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like.

Oral formulations will be in the form of solids or liquids, and may be solutions, syrups, suspensions, tablets, pills, capsules, sustained-release formulations, or powders. Oral formulations include such normally employed excipients as, for example, pharmaceutical grades of mannitol, lactose, starch, magnesium stearate, sodium saccharin, cellulose, magnesium carbonate, and the like.

Topical formulations will generally take the form of suppositories, pessaries, intranasal sprays or aerosols, buccal or sublingual tablets or lozenges. For suppositories or pessaries, traditional binders and excipients may include, for example, polyalkylene glycols or triglycerides; such suppositories or pessaries may be formed from mixtures containing the active ingredient. Other topical formulations may take the form of a lotion, solution, cream, ointment or dusting powder that may optionally be in the form of a skin patch.

In a further aspect, the invention provides a method of treating a proliferative disease selected from gynaecological cancer, such as endometrial or ovarian cancer and polycystic ovary syndrome in which RAGE is over expressed, said method comprising administering to a patient in need thereof an effective amount of a therapeutic agent as described above, or a pharmaceutical composition comprising it, also as described above.

The amount of therapeutic agent administered will vary depending upon factors such as the specific nature of the agent used, the size and health of the patient, the nature of the condition being treated etc. in accordance with normal clinical practice. Typically, a dosage in the range of from 0.01-1000 mg/Kg, for instance from 0.1-10 mg/Kg, would produce a suitable therapeutic or protective effect.

Dosages may be given in a single dose regimen, split dose regimens and/or in multiple dose regimens lasting over several days. Effective daily doses will, however vary depending upon the inherent activity of the therapeutic agent, such variations being within the skill and judgment of the physician.

The therapeutic agent of the present invention may be used in combination with one or more other active agents, such as one or more pharmaceutically active agents. In particular, the applicants have found that anti-hormonal agents such as anti-estrogens and/or selective estrogen receptor modulators such as tamoxifen, may themselves upregulate RAGE expression in gynaecological cancer. Therefore, these agents may act synergistically with the agents of the invention, when the anti-cancer drug carried by the ADC may be the same or different.

Therapeutic agents of the invention may be prepared using conventional methods.

In particular they may be produced by linking together a cell binding agent which binds the RAGE and an anti-cancer drug.

Suitable methods comprise reacting a moiety comprising the linking group with one of either an anti-cancer drug or a cell binding agent, and contacting the product with the other of the anti-cancer drug and the cell binding agent to form the therapeutic agent.

In particular, where the anti-cancer drug is a small molecule, the linking group may be incorporated during the manufacturing process. Thus a particular cytotoxin with a linker attached is Maleimidocaproyl-Val-Cit-PABC-MMAE of structure (I)

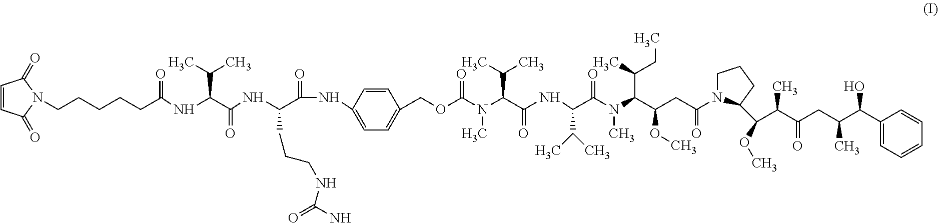 [SMILES: CC[C@H](C)[C@@H]([C@@H](CC(=O)N1CCC[C@H]1[C@H](OC)[C@@H](C)C(=O)C[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

This structure includes the self-immolative linker group maleimidocaproyl-valine-citrulline-p-aminobenzyloxy carbonyl.

Thus in a particular embodiment, in a first step, a linking group is added to the anti-cancer drug and one or more of the resulting product is reacted with the cell binding agent. Suitable reaction conditions for the manufacture of linker attached cytotoxic agents could comprise those described by Doronina et al 2006.¹⁹ Suitable reaction conditions for the attachment of linker attached cytotoxic agents such as maleimidocaproyl-Val-Cit-PABC-MMAE, could also comprise those described by Doronina et al 2006.¹⁹ Specific conditions for each of the stages would be understood or could be determined by the skilled person.

### DETAILED DESCRIPTION OF THE INVENTION

The invention will now be particularly described by way of example with reference to the accompanying diagrammatic drawings in which

FIG. 1 is a graphical representation of the multiligand transmembrane receptor of the immunoglobulin superfamily, RAGE and some of its variant forms;

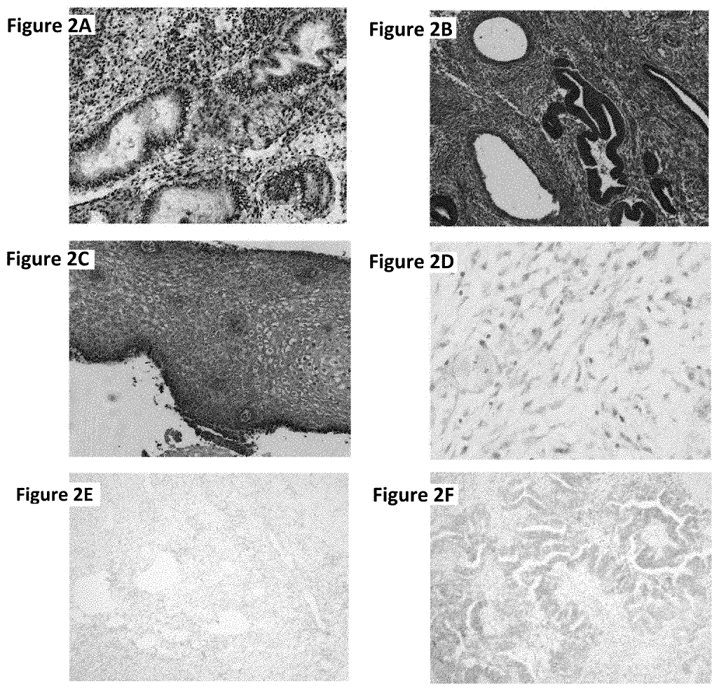

FIGS. 2A-2F show a series of images with RAGE protein expression in biopsies from the endometrium and ovary of a healthy patient and patients with endometrial or ovarian cancer. Endometrial biopsies were collected from the endometrium of a healthy patient (FIG. 2A), and patients with endometrial cancer (FIG. 2B), endometrial hyperplasia (FIG. 2C), or endometriosis (FIG. 2D). Biopsies were fixed and paraffin embedded for analysis of RAGE expression by immunohistochemistry. Further biopsy images show RAGE expression in a healthy ovary (FIG. 2E) and ovarian cancer (endometrioid adenocarcinoma; FIG. 2F).

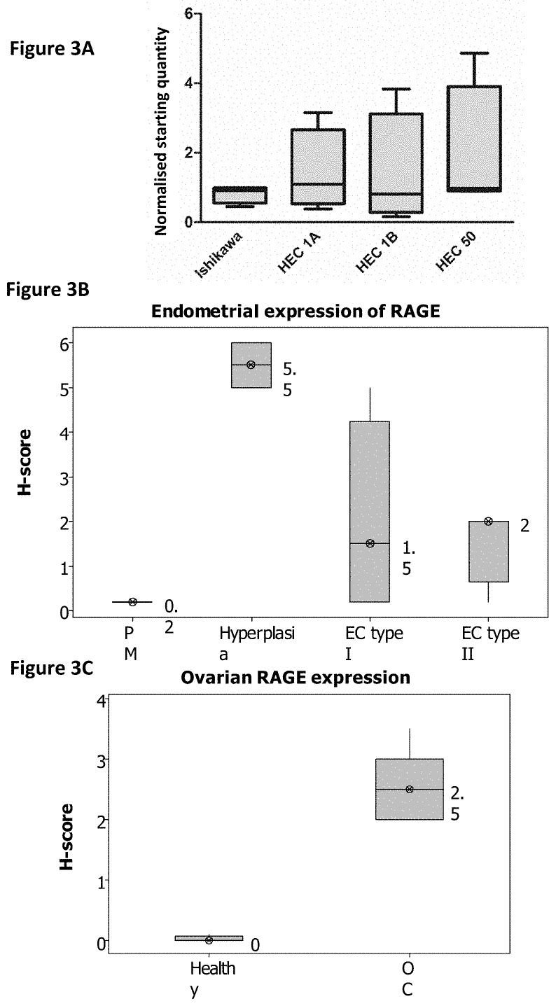

FIGS. 3A-3C are a series of graphs showing (A) the expression of AGER mRNA in four endometrial epithelial cell lines derived from two well-differentiated type I and type II adenocarcinomas; HEC1 (HEC1A, HEC1B, HEC50) and Ishikawa respectively; (B) the results of an immunohistochemistry study showing that endometrial RAGE is overexpressed in hyperplasia and Endometrial cancer; and (C) immunohistochemistry results for RAGE staining in healthy ovary or ovarian cancer (OC) biopsies.

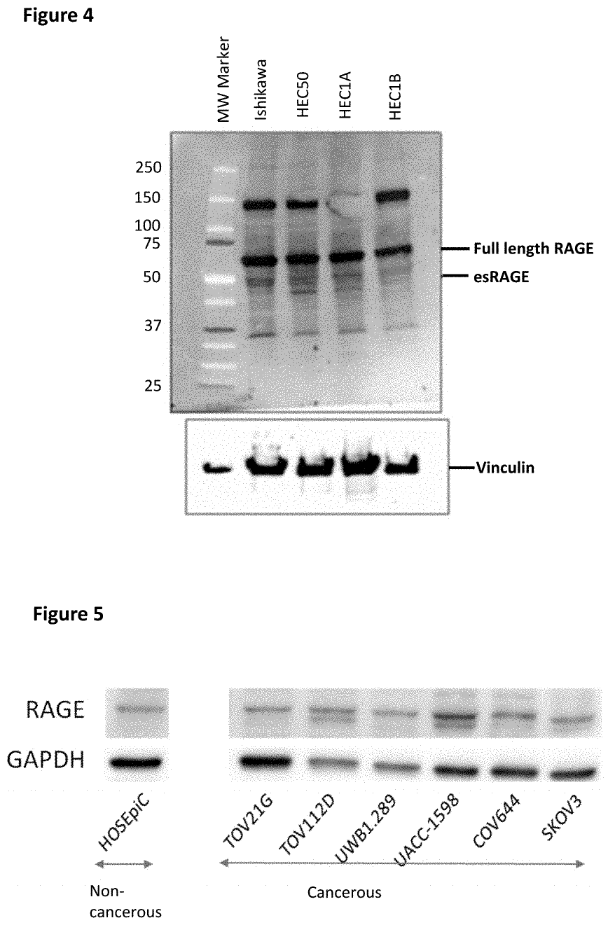

FIG. 4 is a series of Western blots showing RAGE protein expression in the cell lines of FIG. 3.

FIG. 5 is representative Western blots showing expression of RAGE protein in six ovarian cancer cell lines: TOV21G, TOV112D, UWB1.289, UACC-1598, COV644 and SKOV3, and one normal ovarian cell line: HOSEpiC.

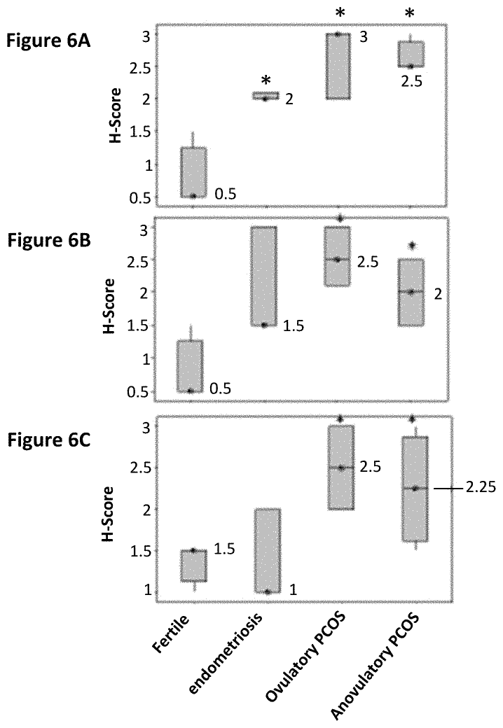

FIGS. 6A-6C are a series of graphs showing RAGE expression scoring (intensity and distribution: H-score) in endometrial biopsy samples, taken during the proliferative phase of the menstrual. RAGE expression scoring (intensity and distribution: H-score) in glandular epithelium (FIG. 6A), luminal epithelium (FIG. 6B) and stroma (FIG. 6C) was performed blind, by three independent reviewers.

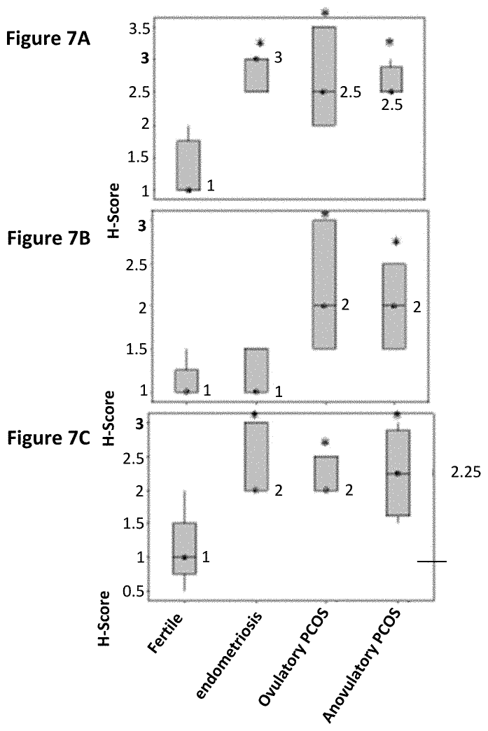

FIGS. 7A-7C are a series of graphs showing RAGE expression scoring (intensity and distribution: H-score) in endometrial biopsy samples, taken during the secretory phase of the menstrual cycle. RAGE expression scoring (intensity and distribution: H-score) in glandular epithelium (FIG. 7A), luminal epithelium (FIG. 7B) and stroma (FIG. 7C) was performed blind, by three independent reviewers.

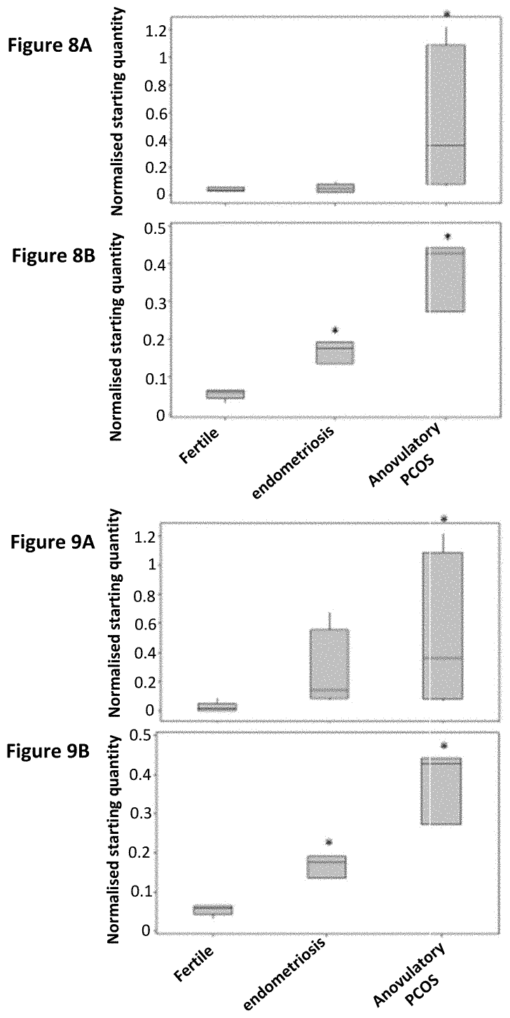

FIGS. 8A-8B are a series of graphs showing AGER mRNA expression in endometrial biopsy samples taken from polycystic ovary syndrome patients during the proliferative phase of the menstrual cycle. Total RNA was extracted from whole endometrial biopsies (FIG. 8A) and endometrial epithelial biopsies (FIG. 8B) for analysis of AGER mRNA expression by quantitative PCR.

FIGS. 9A-9B are a series of graphs showing AGER mRNA expression in endometrial biopsy samples taken from polycystic ovary syndrome patients, during the secretory phase of the menstrual cycle. Total RNA was extracted from whole endometrial biopsies (FIG. 9A) and endometrial epithelial biopsies (FIG. 9B) for analysis of AGER mRNA expression by quantitative PCR.

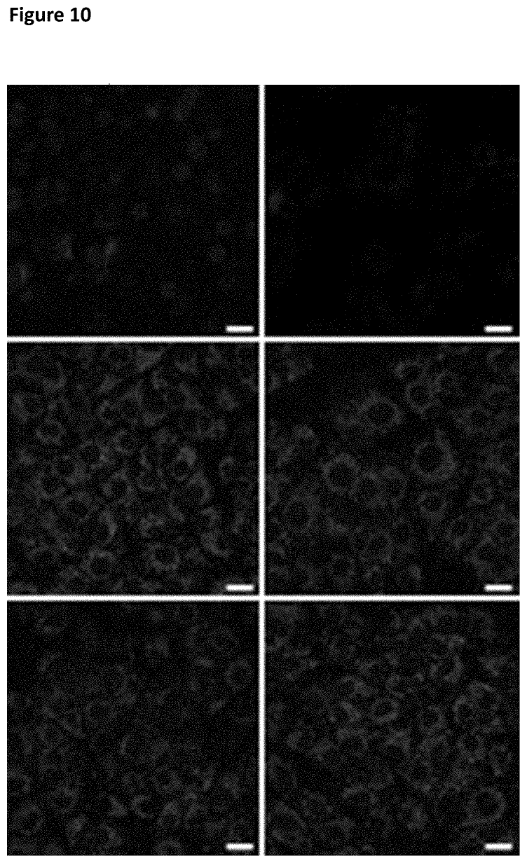

FIG. 10 is a series of representative confocal microscopy images showing the internalisation of anti-RAGE antibody in HEC 1A cells.

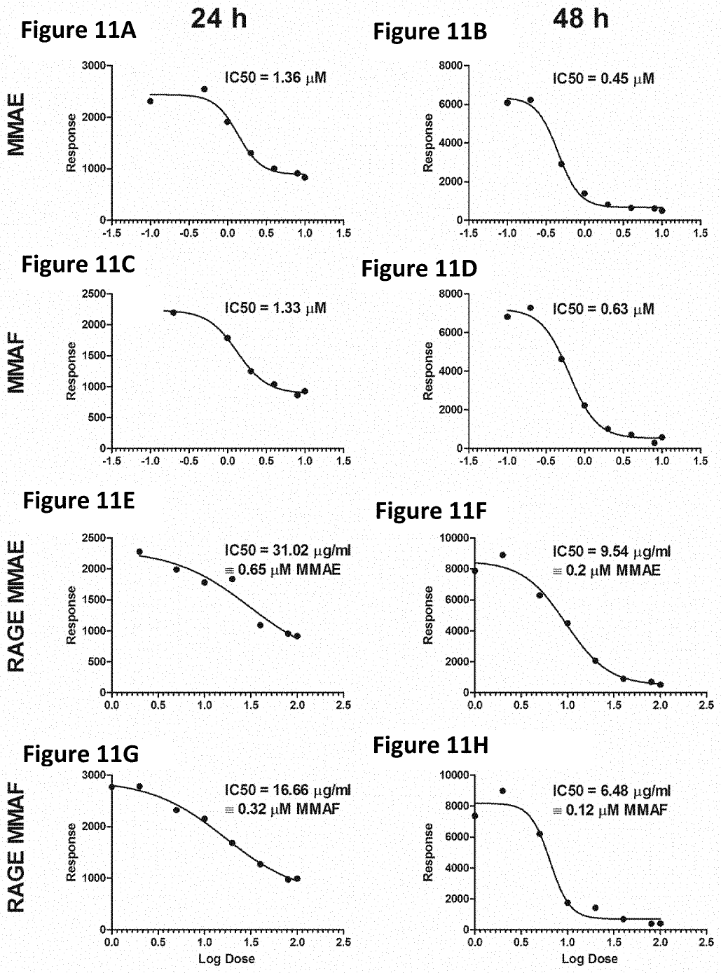

FIGS. 11A-11H is a series of graphs illustrating how delivering cytotoxins in the form of RAGE targeting ADC improves drug potency in endometrial cancer cells. After 24 h treatment, RAGE MMAE (FIG. 11E: IC50=31.02 μg/ml≡0.65 as MMAE μM MMAE) was twice as potent as MMAE alone (FIG. 11A: IC50=1.4 μM), whilst RAGE MMAF (FIG. 11G: IC50=16.66 μg/ml≡0.32 μM MMAF) was four times more potent as MMAF alone (FIG. 11C: IC50=1.3 μM). After 48 h treatment, RAGE MMAE (FIG. 11F: IC50=9.54 μg/ml≡0.2 as MMAE μM MMAE) was again twice as potent as MMAE alone (FIG. 11B: IC50=0.46 μM), and RAGE MMAF (FIG. 11H: IC50=6.48 μg/ml≡0.12 μM MMAF) was five times more potent as MMAF alone (FIG. 11D: IC50=0.63 μM).

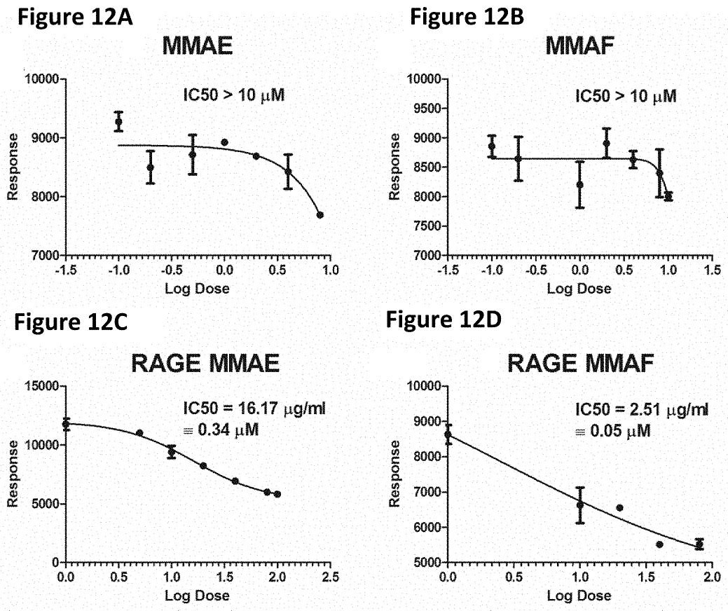

FIGS. 12A-12D is a series of graphs showing how delivering cytotoxins in the form of RAGE targeting ADC improves drug potency in ovarian cancer cells. IC50 concentrations in ovarian (TOV112D) cancer cells after 24 h treatment were 16.67 μg/ml (≡0.65 μM MMAE) for RAGE MMAE (FIG. 12C) and 2.5 μg/ml (≡0.05 μM MMAF) for RAGE MMAF (FIG. 12D). It was not possible to determine IC50 values for the MMAE or MMAF treatments (FIGS. 12A & 12B, respectively) alone in these cells (i.e. the IC50 was greater than the top concentration tested).

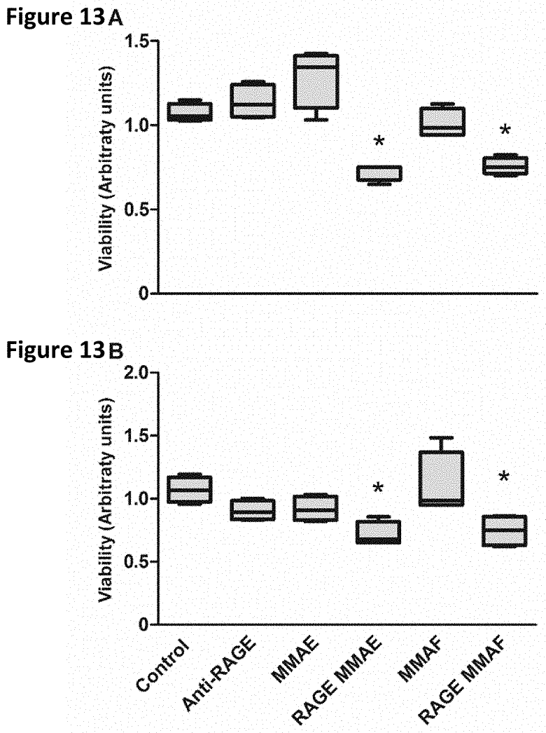

FIGS. 13A-13B is a series of graphs showing that RAGE targeting ADCs are more potent killers of endometrial cancer cells than cytotoxin or antibody treatment alone. Ishikawa (FIG. 13A) or HEC1A (FIG. 13B) cells were seeded into 96-well plates and treated with control medium or medium containing MMAE, MMAF, anti-RAGE antibody, SNIPER MMAE or SNIPER MMAF (shown as RAGE MMAE and RAGE MMAF respectively in FIGS. 13A-13B) for 24 h.

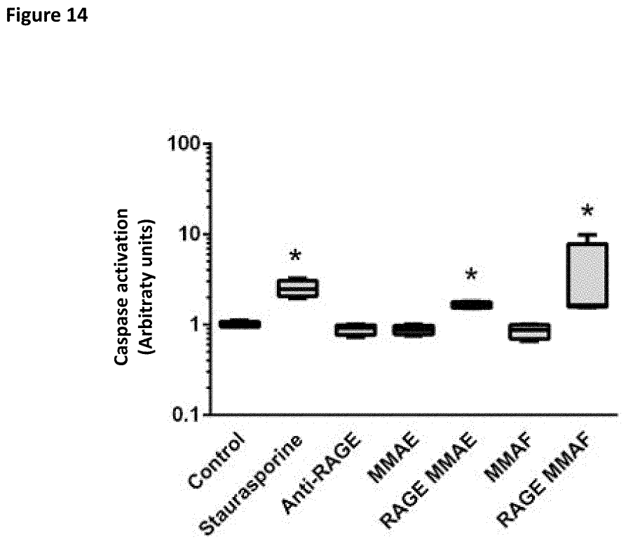

FIG. 14 is a graph illustrating that RAGE targeting ADCs induce apoptosis of endometrial cancer cells.

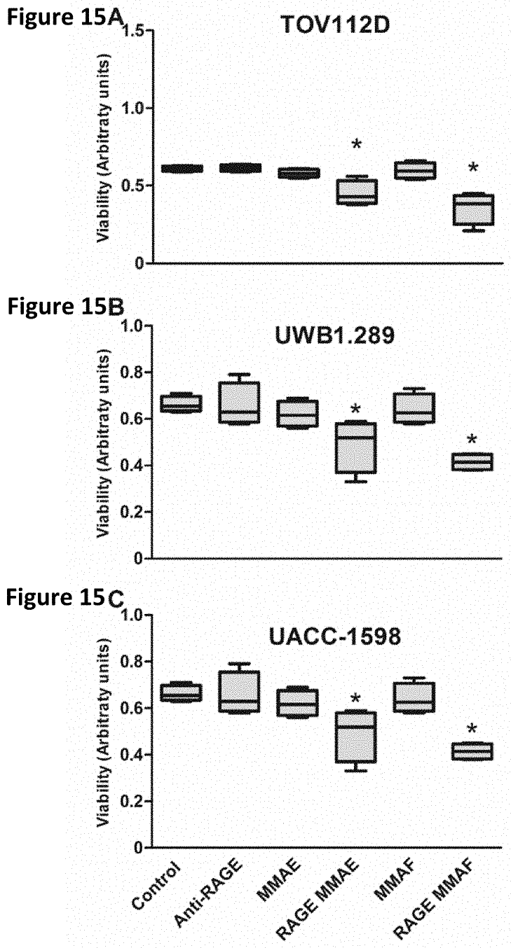

FIGS. 15A-15C is a series of graphs showing that RAGE targeting ADCs are more potent killers of ovarian cancer cells than cytotoxin or antibody treatment alone. Cell viability was determined in TOV112D (FIG. 15A), UWB1.289 (FIG. 15B) and UACC-1595 cells (FIG. 15C).

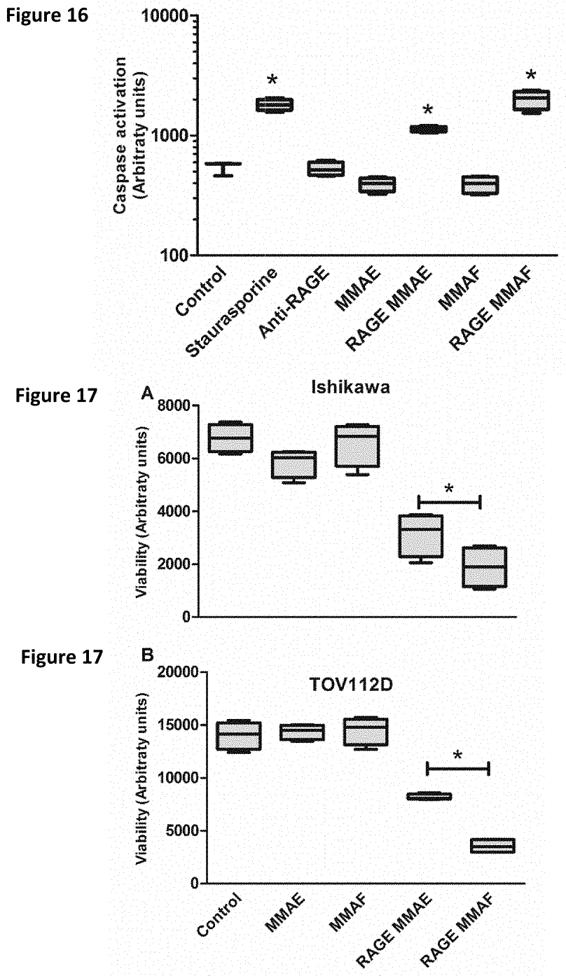

FIG. 16 is a graph showing that RAGE targeting ADCs induce apoptosis of ovarian cancer cells.

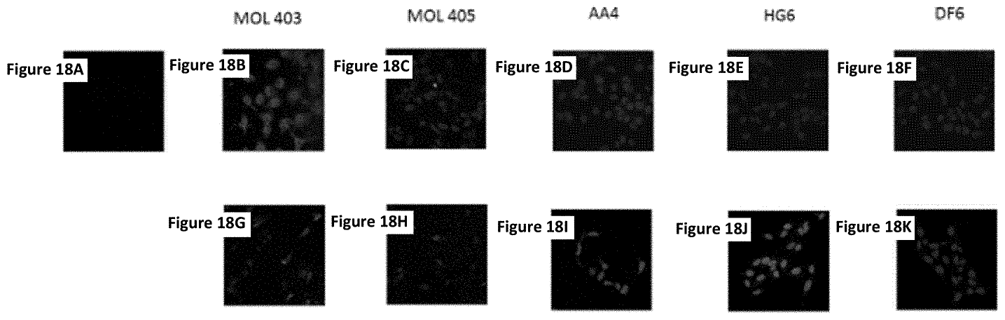

FIGS. 17A-17B is a graph illustrating how using a non-cleavable linker improves ADC potency in endometrial (Ishikawa; FIG. 17A) and ovarian (TOV112D; FIG. 17B) cancer cells.

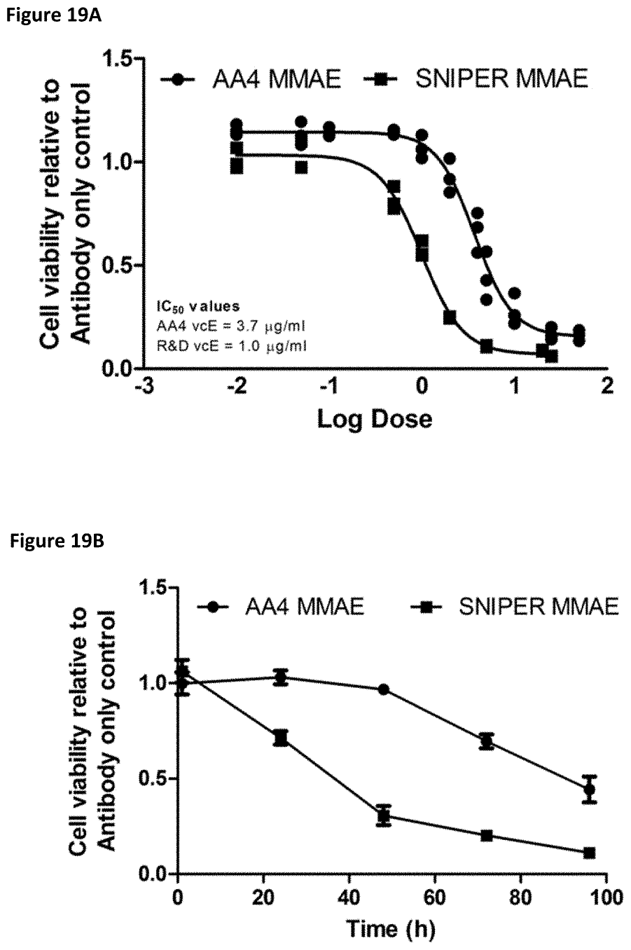

FIGS. 18A-18K is a series of confocal microscopy images showing antibody internalisation in ovarian (FIGS. 18B-18F) and endometrial cancer cells (FIGS. 18G-18K) that have been treated with 5 different anti-RAGE antibodies with a secondary control (FIG. 18A).

FIGS. 19A-19B is a series of graphs showing cell survival rates in HEC 1A cells when treated with ADCs in accordance with the invention. (FIG. 19A) IC50 curves at 96 h, and (FIG. 19B) a time-course graph of cells treated with ADCs (5 μg/ml).

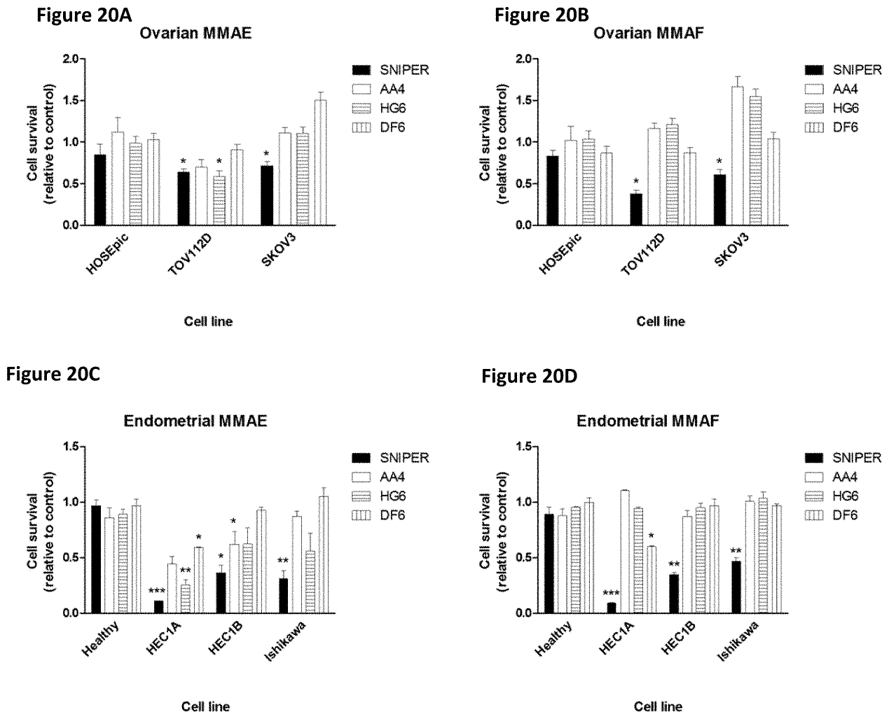

FIGS. 20A-20B is a series of graphs showing cell survival data for a range of cell lines when treated with ADCs in accordance with the invention.

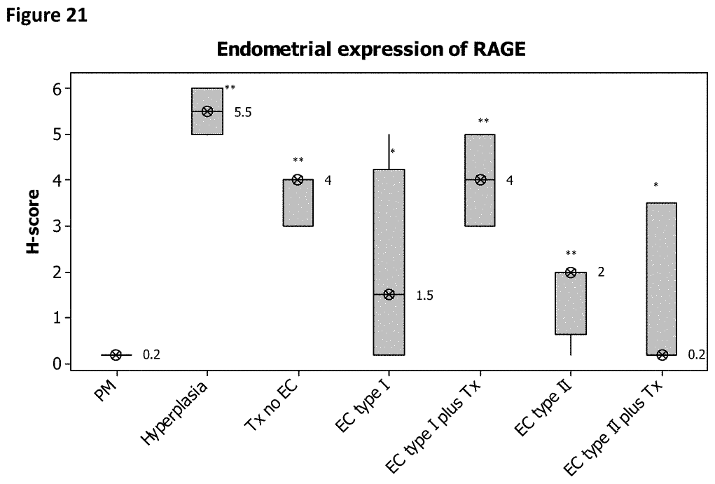

FIG. 21 shows the results of experiments revealing the effect of tamoxifen (Tx) on endometrial expression of RAGE.

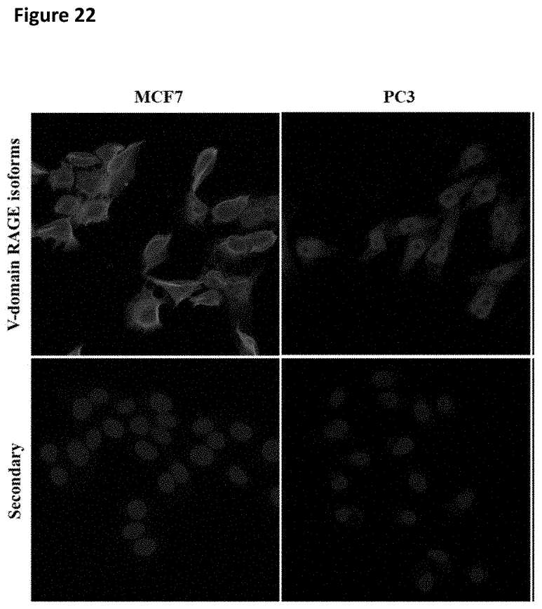

FIG. 22 shows protein expression of RAGE in breast (MCF7) and prostate (PC3) cancer cell lines analysed by confocal microscopy. Cells were then fixed with 4% paraformaldehyde and permeabilised with 1% Triton X-100, followed by 1 hour blocking with 3% BSA. All the treated cells were incubated with the anti-rabbit FITC antibody, overnight. A well incubated only with the secondary antibody was used as non-specific binding control. DAPI was added to all the wells to determine nuclear location.

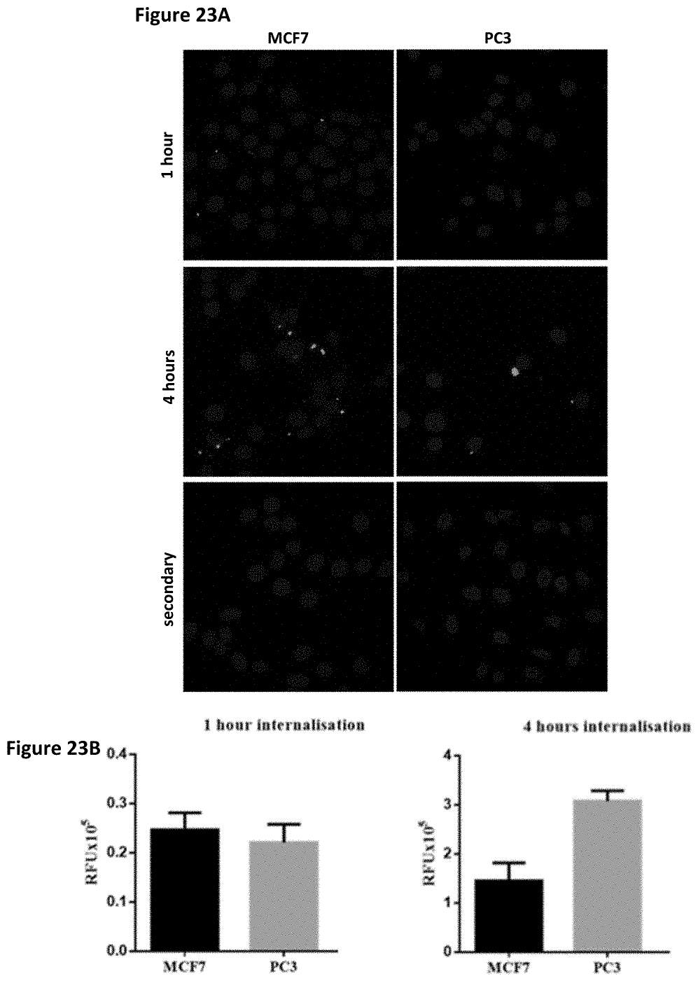

FIGS. 23A-23B shows internalisation of V-region anti-RAGE antibodies in breast (MCF7) and prostate (PC3) cancer cell lines. MCF7 and PC3 cells were seeded in an 8-well chamber slide and incubated with 50 μg/ml anti-RAGE ab (abcam37467) for 1 and 4 hours. The obtained images were analysed using Image J to quantify the fluorescence, and the results were displayed in a graph representing the mean of fluorescence from the three triplicates±StDEV. FIG. 23A shows that, after 1 hour, fluorescent signals corresponding to internalised anti-RAGE antibody are observed. After 4 hours, an increase in antibody-internalisation is observed in all cell lines compared to 1h treatments. FIG. 23B shows values, which are the mean of the fluorescence±StDEV from triplicates; the data was analysed using one-way ANOVA.*p≤0.05.

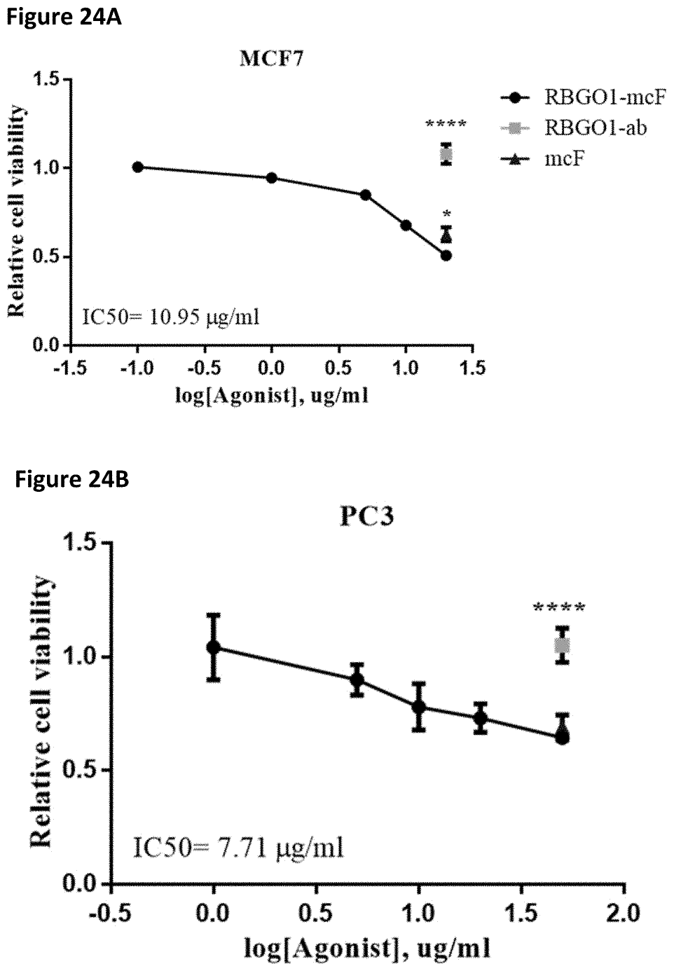

FIGS. 24A-24B shows anti-tumour activity of V-region RAGE-Antibody drug conjugate in in breast (MCF7) and prostate (PC3) cancer cell lines. MCF7 and PC3 cell lines were seeded in a 96 well plate at 1×10⁴ cells per well and grown in striped media for 24 hours. Cells were incubated with different concentration ranges of V-region binding RAGE-MMAF [MCF7 (0.1-20 μg/ml) and PC3 (1-40 μg/ml)] to determine IC50 values. Controls included cells incubated with MMAF, RAGE-antibody alone and DMSO (positive viability control). Cell viability was determined using Promega RealTime-Glo MT reagent. FIG. 24A shows a forty-eight hour treatment of MCF7 with V-region binding RAGE-MMAF revealed an IC50 value equal to 10.95 μg/ml, and induced significant reductions in cell viability compared to MMAF drug (p=0.0120) or RAGE antibody (p<0.0001). FIG. 24B shows the IC50 value of V-region binding RAGE-MMAF in PC3 cells was of 7.71m/ml, with the ADC exhibiting significantly higher activity than the unconjugated antibody (p<0.0001).

### EXAMPLE 1

Expression of RAGE in Gynaecological Cancers and Non Oncological Proliferative conditions

Endometrial biopsies were collected from the endometrium of a healthy patient (FIG. 2A), and patients with endometrial cancer (FIG. 2B), endometrial hyperplasia (FIG. 2C), or endometriosis (FIG. 2D). Biopsies were fixed and paraffin embedded for analysis of RAGE expression by immunohistochemistry.

Further biopsy images show RAGE expression in a healthy ovary (FIG. 2E) and ovarian cancer (endometrioid adenocarcinoma; FIG. 2F). Positive staining was observed in the epithelial cells of the ovarian cystic masses whereas healthy tissue did not express the target.

The expression of AGER mRNA in four endometrial epithelial cell lines derived from two well-differentiated type I and type II adenocarcinomas; HEC1 (HEC1A, HEC1B, HEC50) and Ishikawa respectively, was measured. Epithelial cells were cultured in 6-well plates in control medium. Total RNA was extracted once cells reached confluence for analysis of AGER mRNA expression by quantitative PCR. Data are presented as box plots showing the median (line), 25^{th} and 75^{th} percentiles (box) and 10^{th} and 90^{th} percentile (whiskers), n=5, in FIG. 3A.

In a further experiment, RAGE protein expression was measured in the endometrial biopsies from patients diagnosed with hyperplasia, endometrial cancer Type I or Type II and postmenopausal (PM) controls by immunohistochemistry. Endometrial biopsy samples were grouped as follows: PM (n=25, median=0.2), Hyperplasia (n=21, median=5.5), type I EC (n=18, median=1.5), type II EC (n=17, median=2). IHC samples were scored blind by three independent observers. Values shown are median IHC scores and statistical analysis was performed using a Mann-Whitney test *p<0.05, **p<0.01, compared to PM control.

The results are shown in FIG. 3B. RAGE expression was noted in the membrane and cytoplasm of the tumour cells as well as endometrial cells obtained from hyperplasia patients. PM staining was almost negative. Statistically significant differences in RAGE expression were observed between PM control and all study groups.

RAGE protein expression was also measured by Immunohistochemistry in ovarian biopsies from patients diagnosed with ovarian cancer (n=19) and healthy control patients (n=8). IHC samples were scored blind by three independent observers. The results are shown graphically in FIG. 3C. Values shown are median IHC scores and statistical analysis was performed using a Mann-Whitney test, **p<0.01, compared to healthy control.

RAGE protein expression in the four endometrial cancer epithelial cell lines (HEC1A, HEC1B, HEC50 and Ishikawa), six ovarian cancer epithelial cell lines (TOV21G, TOV112D, UWB1.289, UACC-1598, COV644, SKOV3) and a non-cancerous ovarian cell line (HOSEpiC) were determined by Western blot. Epithelial cells were cultured in 6-well plates in control medium. Protein was extracted once cells reached confluence for analysis of RAGE protein expression. Data are presented as representative Western blots for endometrial and ovarian cell lines, FIGS. 4 and 5, respectively.

These results clearly show that RAGE is upregulated in these gynaecological cancers.

In further experiments, endometrial biopsies were collected from patients during the proliferative phase (n=32) of the menstrual cycle, and subdivided into four groups: fertile (n=9), endometriosis (n=11), ovulatory PCOS (n=12) or anovulatory PCOS (n=14). Biopsies were fixed and paraffin embedded for analysis of RAGE expression by immunohistochemistry. RAGE expression scoring (intensity and distribution: H-score) in glandular epithelium (FIG. 6A), luminal epithelium (FIG. 6B) and stroma (FIG. 6C) was performed blind, by three independent reviewers. The results are shown in FIGS. 6A-6C. Data are presented as box plots showing the median (line), 25^{th} and 75^{th} percentiles (box) and 10^{th} and 90^{th} percentile (whiskers), and analysed by Mann-Whitney U test, values differ from fertile: * P<0.05.

In a separate test, endometrial biopsies were collected from patients during the secretory phase (n=41) of the menstrual cycle, and, as before, subdivided into four groups: fertile (n=12), endometriosis (n=18), ovulatory PCOS (n=11) or anovulatory PCOS (n=14). Biopsies were fixed and paraffin embedded for analysis of RAGE expression by immunohistochemistry.

RAGE expression scoring (intensity and distribution: H-score) in glandular epithelium (FIG. 7A), luminal epithelium (FIG. 7B) and stroma (FIG. 7C) was performed blind, by three independent reviewers. The results are shown in FIG. 7. Data are presented as box plots showing the median (line), 25^{th} and 75^{th} percentiles (box) and 10^{th} and 90^{th} percentile (whiskers), and analysed by Mann-Whitney U test, values differ from fertile: * P<0.05.

In another set of experiments, endometrial biopsies were collected from patients suffering from polycystic ovary syndrome during the proliferative phase and secretive phase (n=32) of the menstrual cycle, and subdivided into three groups: fertile (n=2), endometriosis (n=6) or anovulatory PCOS (n=7). Total RNA was extracted from whole endometrial biopsies (FIGS. 8A and 9A) and endometrial epithelial biopsies (FIGS. 8B and 9B) for analysis of AGER mRNA expression by quantitative PCR. The results are shown in FIGS. 8A-8B and 9A-9B, respectively. Data are presented as box plots showing the median (line), 25^{th} and 75^{th} percentiles (box) and 10^{th} and 90^{th} percentile (whiskers), and analysed by Mann-Whitney U test, values differ from fertile: * P<0.05.

These data show that expression of AGER mRNA and its protein product RAGE is increased in endometrial and ovarian cancers, as well as endometriosis, hyperplasia and polycystic ovary syndrome patients during the proliferative and secretive phase of the menstrual cycle. AGER mRNA expression is also increased in endometrial epithelial cells during the proliferative and secretive phases of the menstrual cycle, and RAGE protein expression is increased in endometrial epithelium during the proliferative phase, and in the endometrial epithelium and stroma during the secretive phase of the menstrual cycle.

### EXAMPLE 2

### Efficacy of RAGE as a Carrier

HEC 1A cells derived from an endometrial adenocarcinoma were cultured on 8-well chamber slides to 80% confluence. Cells were treated with murine, anti-human RAGE (MAB11451; Clone 176902) for the times shown. Cells were fixed and permeabalised, before staining with anti-murine FITC-labelled secondary antibody. Representative images were acquired on a Zeiss 710 confocal microscope and examples are shown in FIG. 10.

This showed that Anti-RAGE antibody is rapidly internalised in cells, making it a good carrier for drugs.

### EXAMPLE 3

### Preparation of Antibody-Drug Conjugates

A murine IgG2B antibody against recombinant human RAGE (R&D Systems Cat No. MAB11451) was reconstituted to 1.59 mg/mL in 10 mM Tris/C1, 2 mM EDTA pH 8.0. The antibody was reduced with 3.5 molar equivalents of 10 mM TCEP:Ab in water for 2 h at 37° C. Without purification the reduced antibody was split in two one each half alkylated with 6.5 molar equivalents of 10 mM vcMMAE or mcMMAF:Ab in DMA (final DMA concentration in the alkylation mixture was 5% v/v) for 2 h at 22° C. Following alkyation N-acetyl cysteine was used to quench any unreacted toxin linker. The conjugates were purified using a HiTrap G25 column equilibrated in 5 mM histidine/C1, 50 mM trehalose, 0.01% w/v olysorbate 20, pH 6.0. The conjugates were analysed by size exclusion chromatography for monomeric content and concentration (using a calibration curve of naked antibody) using size exclusion chromatography. Running conditions: Agilent 1100 HPLC, TOSOH TSKgel G3000SWXL 7.8 mm×30 cm, 5 μm column, 0.5 mL/min in, 0.2 M Potassium Phosphate, 0.25 M Potassium Chloride, 10% IPA, pH 6.95. Drug loading of the conjugates was confirmed using a combination of HIC and reverse phase chromatography. HIC was carried out using a TOSOH Butyl-NPR 4.6 mm×3.5 cm, 2.5 μm column run at 0.8 mL/min with a 12 min linear gradient between A—1.5M (NH4)2SO4, 25 mM NaPi, pH 6.95±0.05 and B—75% 25 mM NaPi, pH 6.95±0.05, 25% IPA. Reverse phase analysis was performed on a Polymer Labs PLRP 2.1 mm×5 cm, 5 μm column run at 1 mL/min at 80° C. with a 25 min linear gradient between 0.05% TFA/H2O and 0.04% TFA/CH3CN. Samples were first reduced by incubation with DTT at pH 8.0 at 37° C. for 15 min. Due to the complex disulphide structure of an IgG2B and hence potential conjugation site variability both the HIC and PLRP chromatographic patterns were too complex to provide an accurate estimation of average drug loading but did confirm a significant level of drug conjugation.

The resulting RAGE ADC was designated ‘SNIPER’.

### EXAMPLE 4

### Effects of ADC on Human Gynaecological Cancer Cells

The cytotoxicity of the SNIPER ADC prepared in Example 3 was tested in a direct comparison to treatment with drug alone or anti-RAGE antibody alone.

Endometrial (Ishikawa) or ovarian (TOV112D) cancer cells were cultured in 96-well plates and treated with an extended concentration range of MMAE, MMAF, RAGE MMAE or RAGE MMAF for 24 or 48 h. Data was analysed by non-linear regression and IC50 concentrations determined for each treatment. After 24 h treatment, RAGE MMAE (FIG. 11E: IC50=31.02 μg/ml≡0.65 as MMAE μM MMAE) was twice as potent as MMAE alone (FIG. 11A: IC50=1.4 μM), whilst RAGE MMAF (FIG. 11G: IC50=16.66 μg/ml≡0.32 μM MMAF) was four times more potent as MMAF alone (FIG. 11C: IC50=1.3 μM). After 48 h treatment, RAGE MMAE (FIG. 11F: IC50=9.54 μg/ml≡0.2 as MMAE μM MMAE) was again twice as potent as MMAE alone (FIG. 11B: IC50=0.46 μM), and RAGE MMAF (FIG. 11H: IC50=6.48 μg/ml≡0.12 μM MMAF) was five times more potent as MMAF alone (FIG. 11D: IC50=0.63 μM).

IC50 concentrations in ovarian (TOV112D) cancer cells after 24 h treatment were 16.67 μg/ml (≡0.65 μM MMAE) for RAGE MMAE (FIG. 12C) and 2.5 μg/ml (≡0.05 μM MMAF) for RAGE MMAF (FIG. 12D). It was not possible to determine IC50 values for the MMAE or MMAF treatments (FIGS. 12A & B, respectively) alone in these cells (i.e. the IC50 was greater than the top concentration tested).

These data demonstrate that delivering cytotoxic agents in the form of a RAGE targeting ADC increases the potency of the drug.

In separate experiments, Ishikawa (FIG. 13A) or HEC1A (FIG. 13B) cells were seeded into 96-well plates and treated with control medium or medium containing MMAE, MMAF, anti-RAGE antibody, SNIPER MMAE or SNIPER MMAF (shown as RAGE MMAE and RAGE MMAF, respectively, in FIGS. 13A-13B) for 24 h. After treatment, cell viability in both cell lines (FIGS. 13A-13B), and cell apoptosis in Ishikawa cells (caspase activation; FIG. 14) were determined by a fluorescence-based cell viability assay (Apotox Glo Triplex assay, Promega) according to the manufacturer's instructions. Data are presented as box plots showing the median (line), 25^{th} and 75^{th} percentiles (box) and 10^{th} and 90^{th} percentile (whiskers), n=4. Data were analysed by ANOVA and Dunnett's pairwise multiple comparison t-test. Values differ from control: * P<0.05. Cell killing and the induction of apoptosis was significantly increased following treatment with ADCs compared to treatment with the drug or antibody alone.

In separate experiments, TOV112D, UWB1.289 or UACC-1595 cells were seeded into 96-well plates and treated with control medium or medium containing MMAE, MMAF, anti-RAGE antibody, SNIPER MMAE or SNIPER MMAF for 24 h. After treatment, cell viability in TOV112D, UWB1.289 and UACC-1595 cells (FIGS. 15A-15C) and the degree of apoptosis in TOV112D cells (caspase activation; FIG. 16) were determined by a fluorescence-based cell viability assay (Apotox Glo Triplex assay, Promega) according to the manufacturer's instructions. Data are presented as box plots showing the median (line), 25^{th} and 75^{th} percentiles (box) and 10^{th} and 90^{th}percentile (whiskers), n=4. Data were analysed by ANOVA and Dunnett's pairwise multiple comparison t-test. Values differ from control: * P<0.05. Cell killing and the induction of apoptosis was significantly increased following treatment with ADCs compared to treatment with the drug or antibody alone.

These data demonstrate that treating cancerous cells with ADCs targeting RAGE is an effective killing strategy that significantly improves the efficacy of the conjugated cytotoxin.

### EXAMPLE 5

### Comparison of Cleavable and Non-Cleavable Linkers

The linkers used in Examples 3 & 4 were directly compared. Ishikawa or TOV112D cells were seeded into 96-well plates and treated with control medium or medium containing MMAE, MMAF, SNIPER MMAE or SNIPER MMAF for 24 h. After treatment, cell viability (FIG. 17) was determined by a fluorescence-based cell viability assay (Apotox Glo Triplex assay, Promega) according to the manufacturer's instructions. Data are presented as box plots showing the median (line), 25^{th} and 75^{th} percentiles (box) and 10^{th} and 90^{th} percentile (whiskers), n=4. Data were analysed by ANOVA and Dunnett's pairwise multiple comparison t-test. Values differ between groups: * P<0.05. SNIPER ADCs were used at 20 μg/ml and drug alone treatments were at equivalent molar concentrations. Cell killing was increased following treatment with ADCs comprising the non-cleavable linker, MMAF, compared to the cleavable linker, MMAE.

These data demonstrate the importance of the correct antibody-linker-drug combination for effective cancer cell killing.

### EXAMPLE 6

### Internalisation of Anti-RAGE Antibodies in Ovarian and Endometrial Cells

Using conventional methods as described for example in Kohler, G. & Milstein, C. Nature 256, 495-497 (1975 and Köhler, G. & Milstein, C. Eur. J. Immun. 6, 511-519 (1976), a series of anti-RAGE antibodies were developed. These were designated AA4, HG6 and DF6. The VH protein sequence of AA4 was as shown in SEQ ID NO 25 and the VL protein sequence of AA4 was as shown in SEQ ID NO 26. The VH protein sequence of HG6 was as shown in SEQ ID NO 25 and the VL protein sequence of HG6 was as shown in SEQ ID NO 26. The VH protein sequence of DF6 was as shown in SEQ ID NO 25 and the VL protein sequence of DF6 was as shown in SEQ ID NO 26.

TOV112D ovarian (FIGS. 18B-18F) or HEC 1A endometrial (FIGS. 18G-18K) cancer cells were cultured on 8-well chamber slides to 80% confluence. Cells were treated with different anti-human RAGE antibodies for 1 h. The antibodies used were MOL403, MOL405, AA4, HG6 and DF6, which bind to the following regions of RAGE, respectively: V-type domain, stub region (SEQ ID No. 24), C-type domain 1, C-type domain 1 and stub region (SEQ ID No. 24). Cells were fixed and permeabilised, before staining with FITC or Alexfluor 488 labelled secondary antibody. Representative images were acquired on a Zeiss 710 confocal microscope and the results are shown in FIG. 18.

All antibodies were internalised in the cells, but internalisation of the MOL403 (V-type domain binding) antibody was assessed as being significantly greater than the other antibodies tested.

### EXAMPLE 7

### Effects of ADC on Healthy and Cancer Cells Over 96 Hours

The methodology of Example 4 was repeated over a 96 h period, using a range of cell lines including endometrial cancer cell lines, Ishikawa, HEC1A, HEC1B, HEC50 and ovarian cancer cells TOV112D as well as healthy endometrial and ovarian cells. The antibody construct used was the SNIPER construct of Example 3.

Results are shown in Table 1 hereinafter. The results show that ADCs are more efficacious after 96 h. In addition, it is clear from Table 1 that the SNIPER-ADC kills endometrial/ovarian cancer cells more effectively than the healthy control cells.

### EXAMPLE 8

### Relative Efficacy of RAGE ADCs Against Gynaecological Cancer Cells

Analysis of the cell killing abilities of ADCs comprising the antibody clones AA4, HG6 and DF6 with MMAE or MMAF, revealed that they were less efficacious than the SNIPER ADC. Antibodies were conjugated to MMAE or MMAF as previously described, and cell viability over a period of 24 to 96 h was determined, also as previously described. Within the concentration ranges tested, 0.01 to 100 μg/ml; it was not possible to determine IC50 values for any of the new antibody clones at the 24, 48 or 72 h time points. After 96 h exposure, IC50 values were determined, showing that the ADCs were less efficacious than the SNIPER ADC at 96 h. An example IC50 comparison graph is shown in FIG. 19A. In addition, comparison of cell killing during the course of the experiment demonstrated that the SNIPER ADC was significantly more effective than the other ADCs (a comparison between AA4 MMAE and SNIPER MMAE is shown in FIG. 19B).

Comparisons of the AA4, HG6 and DF6 ADCs to the SNIPER ADC were made within normal ovarian (HOSEpic) and ovarian cancer (TOV112D and SKOV3) cells, and normal endometrial (Healthy) and endometrial cancer (HEC1A, HEC1B and Ishikawa) cells. Cells were treated for 96 h with 5 μg/ml of each of the ADCs, and cell health monitored as previously described. Within the ovarian cell lines, the SNIPER MMAE ADC was more efficacious compared to the other MMAE ADCs in SKOV3 cells, whilst the SNIPER MMAF ADC was more efficacious in TOV112D and SKOV3 cells (FIG. 20A, B). Data are presented as mean (SEM), and were analysed by ANOVA and Dunnett's pairwise multiple comparison t-test. Values differ from the antibody only control: * P<0.05, ** P<0.01, *** P<0.001.

Within the endometrial cells, the SNIPER MMAE and the SNIPER MMAF ADCs were both significantly more efficacious compared to the other ADCs in HEC1A, HEC1B and Ishikawa cells. There was no significant effect on healthy endometrial cells by any of the ADCs tested (FIG. 20C, 20D).

### EXAMPLE 9

### Tamoxifen Upregulates Endometrial RAGE Expression.

RAGE protein expression was measured by Immunohistochemistry in endometrial biopsies from patients diagnosed with endometrial hyperplasia, Type I or Type II endometrial cancer (EC), postmenopausal controls as well as breast cancer patients taking tamoxifen as part of their treatment that have developed, or not, endometrial cancer. 138 patients were grouped as follows: PM (n=25, median=0.2), Hyperplasia (n=21, median=5.5), type I EC (n=18, median=1.5), type II EC (n=17, median=2), TX no EC (n=19, median=4), type I EC plus TX (n=21, median=4) and type II EC plus TX (n=17, median=0.2).

IHC samples were scored blind by three independent observers. Values shown are median IHC scores and statistical analysis was performed using a Mann-Whitney test *p<0.05, **p<0.01, ***p<0.001, compared to PM control. Table number 2 below shows between group comparisons.

The results are shown in FIG. 21. RAGE expression was noted in the membrane and cytoplasm of tumour cells and endometrial cells obtained from hyperplasia patients. PM staining was almost negative. RAGE expression was also observed in the epithelium and stromal cells of the endometrium from breast cancer patients taken tamoxifen that have not developed Endometrial cancer (Tx no EC). Tamoxifen upregulation of RAGE was also observed in endometrium from EC patients compared to endometrium of EC not taking tamoxifen.

Estrogen receptor a (ER) expression was also measured and was found to be expressed in all groups. Its expression was used as control for tamoxifen action in EC patients.

### EXAMPLE 10

### Anti-RAGE Antibody Drug Conjugates are Effective in Other Cancer Types; Breast and Pancreatic Cancer

The therapeutic potential of RAGE targeted therapies was also evaluated in these cancer types using the anti-RAGE antibody targeting the V-region linked to MMAF.

For confocal microscopy analysis, breast (MCF7) and prostate (PC3) cancer cell lines were seeded in 8-well chamber slides at 4×10⁴ cells per well in growth media for 24 hours. At this point, the cells achieved around 70% confluency. The next day, cells were fixed with 4% paraformaldehyde and permeabilised with 1% Triton X-100, followed by 1 hour blocking with 3% BSA. All the treated cells were incubated with antibody overnight and in the next day, they were washed with PBS and incubated with the anti-goat FITC antibody for 2 hours. A well was incubated with the given secondary antibody as a control for non-specific binding. After this incubation time, cells were washed and DAPI was added to all wells, including an extra control with cells incubated with DAPI only to measure any non-specific fluorescence. The confocal microscopy data represents all the V-domain containing RAGE isoforms. The obtained results showed both cell lines to express RAGE, with the cell line MCF7 exhibiting the strongest RAGE expression, compared to PC3 cells (FIG. 22).

The capacity of RAGE to internalise anti-RAGE antibodies in the cancer cell lines MCF7 and PC3 was also tested. MCF7 and PC3 cells were seeded in 8-well chamber slides at 4×10⁴ cells per well and grown to 70% confluency. Cells were then incubated with the primary antibody targeting the V-domain at a concentration of 50 μg/ml for 1 hour at 4° C., followed by incubations for 1 or 4 hours at 37° C. to assess internalisation. This step was followed by fixation, permeabilisation and blocking of the cells prior to overnight incubation with a secondary antibody anti-rabbit FITC at 4° C. A negative control for non-specific binding was also included per cell line of a well containing secondary antibody only. All cells were washed, and DAPI was added to all the wells including a background fluorescent control with cells incubated with DAPI to account for the 8-well chamber slide signal. The obtained images were analysed using Image J to quantify the fluorescence, and the results were displayed in a graph representing the mean of fluorescence from triplicates±StDEV. Finally, statistical analysis was performed applying one-way ANOVA using the Dunnett's multiple comparison test.

As shown in FIGS. 23A-23B, internalisation of the anti-RAGE antibody was observed after 1 h incubation, which significantly augments after 4 hrs incubation with the antibody in all cell lines (FIGS. 23A-23B). In summary, RAGE is able to internalise antibodies of this receptor in breast and prostate cells.

Finally, the anti-tumour activity of V-region binding RAGE-MMAF was also evaluated in the MCF7 and PC3 cell lines. Cells were seeded in a 96 well plate at 1×10⁴ cells per well and incubated with a range of concentrations of V-region binding RAGE-MMAF added to MCF7 and PC3 (1-40 μg/ml) cell lines to determine the IC50 value of the ADC in each cell line. Controls included wells treated with MMAF or V-region binding RAGE antibody only at the higher ADC concentration used for each cell line. In addition, a control representing the untreated cells was also included. Viability was measured using the Promega RealTime-Glo™ MT viability assay over 96 hrs, taking measurements at 48, 72 and 96 hrs periods. The obtained results were analysed via nonlinear regression to obtain the IC50 value of the V-region binding RAGE-MMAF for each cell line at the different time point mentioned. Data was evaluated via one-way ANOVA using the Dunnett's multiple comparison test. As shown in FIG. 24A in the cell line MCF7, revealed an IC50 value equal to 10.95 μg/ml after 48 hours treatment with V-region binding RAGE-MMAF demonstrating the ADC's anti-tumour activity against breast cancer cells. In addition, incubations with MMAF and V-region binding RAGE antibody reduced the biological activity of MCF7 cells significantly compared to the untreated control (p=<0.0001). V-region binding RAGE antibody incubations did not affect the cell viability compared to untreated cells. Finally, the higher dose of V-region binding RAGE-MMAF used (20 μg/ml) was more potent at killing MCF7 cell lines than either the V-region binding RAGE-antibody alone or the MMAF drug (p<0.0001 & p=0.0120, respectively), evaluated at the same concentration used in the ADC, thus, indicating an increased in cytotoxicity of the drug on conjugation to the targeting antibody. Therefore, V-region binding RAGE-MMAF is able to have an antitumour effect in MCF7 cells, suggesting RAGE expression provides a therapeutic advantage in breast cancer cells (FIG. 24A).

In PC3 cells, V-region binding RAGE-MMAF exhibited an IC50 value of 7.71 μg/ml of the same magnitude as reported for endometrial, ovarian and breast cancer cells tested. The controls used to measure the potency and the functionality of the RAGE-ADC (V-region binding RAGE alone, and MMAF alone) were prepared at the same proportion and amount and that one found in the V-region binding RAGE-MMAF at 40m/ml, which was the highest dose used to perform the standard curve. The results revealed that the drugs MMAF and V-region binding RAGE-MMAF significantly reduced the viability of PC3 cancer cells compared to the untreated cells (p=0.0013 & p=0.0004, respectively). Incubations with V-region binding RAGE antibody alone did not affect the cell viability compared to untreated cells. Finally, the ADC was more potent killing the PC3 prostate cancer cells than each of its components in isolation with significant differences observed in viability between cells treated with V-region binding RAGE-MMAF and V-region binding RAGE antibody alone (p<0.0001) (FIG. 24B). In summary, RAGE is overexpressed in breast and prostate cell lines, and it is able to internalise antibodies recognising its V-domain. The evaluation of the IC50 value over time in both MCF7 and PC3 cell lines showed that the anti-tumour activity of V-region binding RAGE-MMAF is directly proportional to the exposure time in breast and prostate cells (FIGS. 24A-24B). These experiments provided indicate suitability of RAGE-ADC therapy to treat other cancers that exhibit RAGE overexpression.

TABLE 1
IC50

| | | SNIPER-MMAE (μg/ml) | | | | | | SNIPER-MMAF(μg/ml) | | | | |
|---|---|---|---|---|---|---|---|---|---|---|---|---|
| | Cell | [Drug only equivalent, μM] | | | | | | [Drug only equivalent, μM] | | | | |
| Tissue | line | 24 h | | 48 h | | 96 h | | 24 h | | 48 h | | 96 h |
| Endometrium | Healthy | ND | | 10.72 | [0.22] | 15.19 | [0.31] | ND | | 7.25 | [0.14] | 4.17 [0.08] |
| | HEC1A | 10.34 | [0.22] | 4.69 | [0.1] | 1.02 | [0.02] | 24.11 | [0.46] | 0.81 | [0.02] | 0.74 [0.02] |
| | HEC1B | 29.04 | [0.61] | 8.65 | [0.18] | 5.67 | [0.12] | ND | | 1.96 | [0.04] | 1.27 [0.02] |
| | HEC50 | ND | | 7.64 | [0.16] | 2.18 | [0.05] | 17.82 | [0.33] | 0.86 | [0.02] | 0.94 [0.02] |
| | Ishikawa | 31.02 | [0.65] | 9.54 | [0.2] | 3.86 | [0.08] | 16.7 | [0.32] | 6.48 | [0.12] | 2.42 [0.04] |

| Ovary | Healthy | ND | | ND | | 41.02 | [0.86] | ND | 14.36 | [0.27] | 4.87 [0.09] |
|---|---|---|---|---|---|---|---|---|---|---|---|
| | TOV112D | 22.6 | [0.47] | 16.17 | [0.34] | 0.54 | [0.01] | ND | 2.51 | [0.05] | 0.59 [0.01] |

ND = not determined within the ADC concentration range used (0.01 to 100 μg/ml)

TABLE 2

| Comparisons | EC type | | | | | |
|---|---|---|---|---|---|---|
| RAGE | II plus | EC | EC type | TX no | Hyper- | |
| expression | Tx | type I | II | EC | plasia | PM |
| EC type I plus | 0.0320 | 0.0500 | 0.0003 | 0.5419 | 0.0093 | 0.0002 |
| Tx | | | | | | |
| EC type II plus | | 0.3572 | 0.4442 | 0.0074 | 0.0007 | 0.0450 |
| Tx | | | | | | |
| EC type I | | | 0.8008 | 0.2476 | 0.0015 | 0.0301 |
| EC type II | | | | 0.0003 | 0.0014 | 0.0072 |
| TX no EC | | | | | 0.0011 | 0.0003 |
| Hyperplasia | | | | | | 0.0011 |

TABLE 3
Comparison table of the in vitro IC50 values of V-region binding
RAGE-MMAF evaluated in human breast and pancreatic cancer cells
during 96 hours incubation. The IC50 value of RAGE-MMAF in each
cell line decreased in a time dependent manner. The values given
are the average from the three triplicates ± StDEV.

| | V-region RAGE-MMAF IC50 (μg/ml) | | |
|---|---|---|---|
| Cancer Cell | [Drug equivalent in μM] | | |
| Lines | 48 hrs | 72 hrs | 96 hrs |
| MCF7 | 10.95 [0.27 μM] | 9.28 [0.23 μM] | 8.15 [0.20 μM] |
| PC3 | 7.71 [0.19 μM] | 7.6 [0.19 μM] | 5.31 [0.13 μM] |

Data was analysed using non-linear regression and one-way ANOVA
*p ≤ 0.05,
**p ≤ 0.01,
***p ≤ 0.001,
****p ≤ 0.0001.

### Sequences Referred to Herein

| SEQ | |
|---|---|
| ID | |
| NO | |
| 1 | MAAGTAVGAWVLVLSLWGAVVGAQNITARIGEPLVLKCKGAPKKP |
| | PQRLEWKLNTGRTEAWKVLSPQGGGPWDSVARVLPNGSLFLPAVG |
| | IQDEGIFRCQAMNRNGKETKSNYRVRVYQIPGKPEIVDSASELTA |
| | GVPNKVGTCVSEGSYPAGTLSWHLDGKPLVPNEKGVSVKEQTRRH |
| | PETGLFTLQSELMVTPARGGDPRPTFSCSFSPGLPRHRALRTAPI |
| | QPRVWEPVPLEEVQLVVEPEGGAVAPGGTVTLTCEVPAQPSPQIH |
| | WMKDGVPLPLPPSPVLILPEIGPQDQGTYSCVATHSSHGPQESRA |
| | VSISIIEPGEEGPTAGSVGGSGLGTLALALGILGGLGTAALLIGV |
| | ILWQRRQRRGEERKAPENQEEEEERAELNQSEEPEAGESSTGGP |
| 2 | MAAGTAVGAWVLVLSLWGAVVGAQNITARIGEPLVLKCKGAPKKP |
| | PQRLEWKLNTGRTEAWKVLSPQGGGPWDSVARVLPNGSLFLPAVG |
| | IQDEGIFRCQAMNRNGKETKSNYRVRVYQIPGKPEIVDSASELTA |
| | GVPNKVGTCVSEGSYPAGTLSWHLDGKPLTRRHPETGLFTLQSEL |
| | MVTPARGGDPRPTFSCSFSPGLPRHRALRTAPIQPRVWEPVPLEE |
| | VQLVVEPEGGAVAPGGTVTLTCEVPAQPSPQIHWMKDGVPLPLPP |
| | SPVLILPEIGPQDQGTYSCVATHSSHGPQESRAVSISIIEPGEEG |
| | PTAGEGFDKVREAEDSPQHM |
| 3 | MAAGTAVGAWVLVLSLWGAVVGAQNITARIGEPLVLKCKGAPKKP |
| | PQRLEWKLNTGRTEAWKVLSPQGGGPWDSVARVLPNGSLFLPAVG |
| | IQDEGIFRCQAMNRNGKETKSNYRVRVYQIPGKPEIVDSASELTA |
| | GVPNKVVEESRRSRKRPCEQEVGTCVSEGSYPAGTLSWHLDGKPL |
| | VPNEKGVSVKEQTRRHPETGLFTLQSELMVTPARGGDPRPTFSCS |
| | FSPGLPRHRALRTAPIQPRVWEPVPLEEVQLVVEPEGGAVAPGGT |
| | VTLTCEVPAQPSPQIHWMKDGVPLPLPPSPVLILPEIGPQDQGTY |
| | SCVATHSSHGPQESRAVSISIIEPGEEGPTAGSVGGSGLGTLALA |
| | LGILGGLGTAALLIGVILWQRRQRRGEERKAPENQEEEEERAELN |
| | QSEEPEAGESSTGGP |
| 4 | MAAGTAVGAWVLVLSLWGAVVGAQNITARIGEPLVLKCKGAPKKP |
| | PQRLEWKLGGGPWDSVARVLPNGSLFLPAVGIQDEGIFRCQAMNR |
| | NGKETKSNYRVRVYQIPGKPEIVDSASELTAGVPNKVGTCVSEGS |
| | YPAGTLSWHLDGKPLVPNEKGVSVKEQTRRHPETGLFTLQSELMV |
| | TPARGGDPRPTFSCSFSPGLPRHRALRTAPIQPRVWEPVPLEEVQ |
| | LVVEPEGGAVAPGGTVTLTCEVPAQPSPQIHWMKDGVPLPLPPSP |
| | VLILPEIGPQDQGTYSCVATHSSHGPQESRAVSISIIEPGEEGPT |
| | AGSVGGSGLGTLALALGILGGLGTAALLIGVILWQRRQRRGEERK |
| | APENQEEEEERAELNQSEEPEAGESSTGGP |
| 5 | MAAGTAVGAWVLVLSLWGAVVGAQNITARIGEPLVLKCKGAPKKP |
| | PQRLEWKLNTGRTEAWKVLSPQGGGPWDSVARVLPNGSLFLPAVG |
| | IQDEGIFRCQAMNRNGKETKSNYRVRVYQIPGKPEIVDSASELTA |
| | GVPNKVVEESRRSRKRPCEQEVGTCVSEGSYPAGTLSWHLDGKPL |
| | VPNEKGVSVKEQTRRHPETGLFTLQSELMVTPARGGDPRPTFSCS |
| | FSPGLPRHRALRTAPIQPRVWEPVPLEEVQLVVEPEGGAVAPGGT |
| | VTLTCEVPAQPSPQIHWMKDGVPLPLPPSPVLILPEIGPQDQGTY |
| | SCVATHSSHGPQESRAVSISIIEPGEEGPTAGEGFDKVREAEDSP |
| | QHM |
| 6 | MAAGTAVGAWVLVLSLWGAVVGAQNITARIGEPLVLKCKGAPKKP |
| | PQRLEWKLNTGRTEAWKVLSPQGGGPWDSVARVLPNGSLFLPAVG |
| | IQDEGIFRCQAMNRNGKETKSNYRVRVYQIPGKPEIVDSASELTA |
| | GVPNKVGTCVSEGSYPAGTLSWHLDGKPLVPNEKGVSVKEQTRRH |
| | PETGLFTLQSELMVTPARGGDPRPTFSCSFSPGLPRHRALRTAPI |
| | QPRVWEPVPLEEVQLVVEPEGGAVAPGGTVTLTCEVPAQPSPQIH |
| | WMKDGLRTREPTAVWPPIPATGPRKAVLSASASSNQARRGQLQVR |
| | GLIKSGKQKIAPNTCDWGDGQQERNGRPQKTRRKRR |
| 7 | MAAGTAVGAWVLVLSLWGAVVGAQNITARIGEPLVLKCKGAPKKP |
| | PQRLEWKLNTGRTEAWKVLSPQGGGPWDSVARVLPNGSLFLPAVG |
| | IQDEGIFRCQAMNRNGKETKSNYRVRVYQIPGKPEIVDSASELTA |
| | GVPNKVGTCVSEGSYPAGTLSWHLDGKPLVPNEKGVSVKEQTRRH |
| | PETGLFTLQSELMVTPARGGDPRPTFSCSFSPGLPRHRALRTAPI |
| | QPRVWEPVPLEEVQLVVEPEGGAVAPGGTVTLTCEVPAQPSPQIH |
| | WMKDGVPLPLPPSPVLILPEIGPQDQGTYSCVATHSSHGPQESRA |
| | VSISIIEPGEEGPTAGEGFDKVREAEDSPQHM |
| 8 | MAAGTAVGAWVLVLSLWGAVVGAQNITARIGEPLVLKCKGAPKKP |
| | PQRLEWKLGGGPWDSVARVLPNGSLFLPAVGIQDEGIFRCQAMNR |
| | NGKETKSNYRVRVYQIPGKPEIVDSASELTAGVPNKVGTCVSEGS |
| | YPAGTLSWHLDGKPLVPNEKGVSVKEQTRRHPETGLFTLQSELMV |
| | TPARGGDPRPTFSCSFSPGLPRHRALRTAPIQPRVWEPVPLEEVQ |
| | LVVEPEGGAVAPGGTVTLTCEVPAQPSPQIHWMKDVSDLERGAGR |
| | TRRGGANCRLCGRIRAGNSSPGPGDPGRPGDSRPAHWGHLVAKAA |
| | TPRRGEEGPRKPGGRGGACRTESVGGT |
| 9 | MAAGTAVGAWVLVLSLWGAVVGAQNITARIGEPLVLKCKGAPKKP |
| | PQRLEWKLNTGRTEAWKVLSPQGGGPWDSVARVLPNGSLFLPAVG |
| | IQDEGIFRCQAMNRNGKETKSNYRVRVYQIPGKPEIVDSASELTA |
| | GVPNKVGTCVSEGSYPAGTLSWHLDGKPLVPNEKGVSVKEQTRRH |
| | PETGLFTLQSELMVTPARGGDPRPTFSCSFSPGLPRHRALRTAPI |
| | QPRVWEPVPLEEVQLVVEPEGGAVAPGGTVTLTCEVPAQPSPQIH |
| | WMKDNQARRGQLQVRGLIKSGKQKIAPNTCDWGDGQQERNGRPQK |
| | TRRKRRSVQN |
| 10 | MAAGTAVGAWVLVLSLWGAVVGAQNITARIGEPLVLKCKGAPKKP |
| | PQRLEWKLNTGRTEAWKVLSPQGGGPWDSVARVLPNGSLFLPAVG |
| | IQDEGIFRCQAMNRNGKETKSNYRVRVYQIPGKPEIVDSASELTA |
| | GVPNKVGTCVSEGSYPAGTLSWHLDGKPLVPNEKGVSVKEQTRRH |
| | PETGLFTLQSELMVTPARGGDPRPTFSCSFSPGLPRHRALRTAPI |
| | QPRVWEPVPLEEVQLVVEPEGGAVAPGGTVTLTCEVPAQPSPQIH |
| | WMKDGVPLPLPPSPVLILPEIGPQDQGTYSCVATHSSHGPQESRA |
| | VSISIIEPGEEGPTAGEGFDKVREAEDSPQHM |
| 11 | MAAGTAVGACASGGGPIGGGARRWSSSSWWNRNPDL |
| 12 | MAAGTAVGAWVLVLSLWGAVVGAQNITARIGEPLVLKCKGAPKKP |
| | PQRLEWKLNTGRTEAWKVLSPQGGGPWDSVARVLPNGSLFLPAVG |
| | IQDEGIFRCQAMNRNGKETKSNWWWSQKVEQ |
| 13 | MAAGTAVGAWVLVLSLWGAVVGAQNITARIGEPLVLKCKGAPKKP |
| | PQRLEWKLNTGRTEAWKVLSPQGGGPWDSVARVLPNGSLFLPAVG |
| | IQDEGILGGLGTAALLIGVILWQRRQRRGEERKAPENQEEEEERA |
| | ELNQSEEPEAGESSTGGP |
| 14 | MVTPARGGDPRPTFSCSFSPGPPRHRALRTAPIQPRVWEPVPLEE |
| | VQLVVEPEGGAVAPGGTVTLTCEVPAQPSPQIHWMKDGVPLPLPP |
| | SPVLILPEIGPQDQGTYSCVATHSSHGPQESRAVSISIIEPGEEG |
| | PTAGEGFDKVREAEDSPQHM |
| 15 | MERRPSPTTESVSTSLRTFTITASDWIFPPSEIPGKPEIVDSASE |
| | LTAGVPNKVGTCVSEGSYPAGTLSWHLDGKPLVPNEKGVSVKEQT |
| | RRHPETGLFTLQSELMVTPARGGDPRPTFSCSFSPGLPRHRALRT |
| | APIQPRVWEPVPLEEVQLVVEPEGGAVAPGGTVTLTCEVPAQPSP |
| | QIHWMKDGVPLPLPPSPVLILPEIGPQDQGTYSCVATHSSHGPQE |
| | SRAVSISIIEPGEEGPTAGEGFDKVREAEDSPQHM |
| 16 | MNRNGKETKSNYRVRVYQIPGKPEIVDSASELTAGVPNKVGTCVS |
| | EGSYPAGTLSWHLDGKPLVPNEKGVSVKEQTRRHPETGLFTLQSE |
| | LMVTPARGGDPRPTFSCSFSPGLPRHRALRTAPIQPRVWEPVPLE |
| | EVQLVVEPEGGVVAPGGTVTLTCEVPAQPSPQIHWMKDGVPLPLP |
| | PSPVLILPEIGPQDQGTYSCVATHSSHGPQESRAVSISIIEPGEE |
| | GPTAGEGFDKVREAEDSPQHM |
| 17 | MERRPSPTTESVSTSLRTFTITASDWIFPPSEIPGKPEIVDSASE |
| | LTAGVPHKVGTCVSEGSYPAGTLSWHLDGKPLVPNEKGVSVKEQT |
| | RRHPETGLFTLQSELMVTPARGGDPRPTFSCSFSPGLPRHRALRT |
| | APIQPRVWEPVPLEEVQLVVEPEGGAVAPGGTVTLTCEVPAQPSP |
| | QIHWMKDGVPLPLPPSPVLILPEIGPQDQGTYSCVATHSSHGPQE |
| | SRAVSISIIEPGEEGPTAGEGFDKVREAEDSPQHM |
| 18 | MGSPWCLMRRGVSVKEQTRRHPETGLFTLQSELMVTPARGGDPRP |
| | TFSCSFSPGLPRHRALRTAPIQPRVWEPVPLEEVQLVVEPEGGAV |
| | APGGTVTLTCEVPAQPSPQIHWMKDGVPLPLPPSPVLILPEIGPQ |
| | DQGTYSCVATHSSHGPQESRAVSISIIEPGEEGPTAGSVGGSGLG |
| | TLALALGILGGLGTAALLIGVILWQRRQRRGEERKAPENQEEEEE |
| | RAELNQSEEPEAGESSTGGP |
| 19 | MNRNGKETKSNYRVRVYQIPGKPEIVDSASELTAGVPNKVGTCVS |
| | EGSYPAGTLSWHLDGKPLVPNEKGVSVKEQTRRHPETGLFTLQSE |
| | LMVTPARGGDPRPTFSCSFSPGLPRHRALRTAPIQPRVWEPVPLE |
| | EVQLVVEPEGGAVAPGGTVTLTCEVPAQPSPQIHWMKDGVPLPLP |
| | PSPVLILPEIGPQDQGTYSCVATHSSHGPQESRAVSISIIEPGEE |
| | GPTAGEGFDKVREAEDSPQHM |
| 20 | MAAGTAVGAWVLVLSLWGAVVGAQNITARIGEPLVLKCKGAPKKP |
| | RPQLEWKLNTGRTEAWKVLSPQGGGPWDSVARVLPNGSLFLPAVG |
| | IQDEGIFRCQAMNRNGKETKSNYRVRVYQIPGKPEIVDSASELTA |
| | GVPNKVGTCVSEGSYPAGTLSWHLDGKPLVPNEKGVSVKEQTRRH |
| | PETGLFTLQSELMVTPARGGDPRPTFSCSFSPGLPRHRALRTAPI |
| | QPRVWGEHRWGGPQAHVSTFWKSDP |
| 21 | MAAGTAVGAWVLVLSLWGAVVGAQNITARIGEPLVLKCKGAPKKP |
| | PQRLEWKLNTGRTEAWKVLSPQGGGPWDSVARVLPNGSLFLPAVG |
| | IQDEGIFRCQAMNRNGKETKSNWWWSQKVEQ |
| 22 | MAAGTAVGAWVLVLSLWGAVVGAQNITARIGEPLVLKCKGAPKKP |
| | PQRLEWKLNTGRTEAWKVLSPQGGGPWDSVARVLPNGSLFLPAVG |
| | IQDEGIFRCQAMNRNGKETKSNYRVRVYQIPGKPEIVDSASELTA |
| | GVPNKVGTCVSEGSYPAGTLSWHLDGKPLVPNEKGVSVKEQTRRH |
| | PETGLFTLQSELMVTPARGGDPRPTFSCSFSPGLPRHRALRTAPI |
| | QPRVWEPVPLEEVQLVVEPEGGAVAPGGTVTLTCEVPAQPSPQIH |
| | WMKDGVPLPLPPSPVLILPEIGPQDQGTYSCVATHSSHGPQESRA |
| | VSISIIEPGEEGPTAGSVGGSGLGTLALALGILGGLGTAALLIGV |
| | ILWQRRQRRAELNQSEEPEAGESSTGGP |
| 23 | MNRNGKETKSNYRVRVYQIPGKPEIVDSASELTAGVPNKVGTCVS |
| | EGSYPAGTLSWHLDGKPLVPNEKGVSVKEQTRRHPETGLFTLQSE |
| | LMVTPARGGDPRPTFSCSFSPGLPRHRALRTAPIQPRVWGEHRWG |
| | GPQAHVSTFWKSDP |
| 24 | SISIIEPGEEGPTAGSVGGSGLGTLALA |
| 25 | QVQLQQSGAELVKPGASVKLSCKTSGYTFTNYYIYWVIQRPGHGL |
| | GEWIEINPSNGGTNFSERFKSRAKLTVDKPSSTAYMQLSSLTSDD |
| | SAVYYCTTNFDYWGQGSTLTVSS |
| 26 | DVLMTQTPLSLPVSLGDQASMSCRSSQNIVHNNGNTYLQWYLQKP |
| | GQSPKLLIYQVSNRFFGVPDRFSGSGSGTDFTLKISRVEAEDLGV |
| | YYCFQGSHLPLTFGAGTKLELK |
| 27 | QVQLLQPGAELVRPGASVRLSCKASGYTFTSYWINWVKQRPGQGL |
| | EWIGNIYPSDSYTNYNQKFKDKATLTVDKSSSTAYMQLSSPTSED |
| | SAVYYCAREGYWGQGTLVTVSA |
| 28 | ELVMTQSPLTLSVTIGQPASISCKSGQSLLYSNGKTYLYWLLQRP |
| | GQSPKRLIYLVSKLDSGVPDRFTGSGSGTDFTLKISRVEAEDLGV |
| | YYCVQGTHFPYTFGGGTKLEIK |

### REFERENCES

1 Neeper, M. et al. Cloning and expression of a cell surface receptor for advanced glycosylation end products of proteins. J Biol Chem 267, 14998-15004 (1992).
2 Sugaya, K. et al. Three genes in the human MHC class III region near the junction with the class II: gene for receptor of advanced glycosylation end products, PBX2 homeobox gene and a notch homolog, human counterpart of mouse mammary tumor gene int-3. Genomics 23, 408-419, doi:S0888754384715175 [pii] (1994).
3 Fritz, G. RAGE: a single receptor fits multiple ligands. Trends Biochem Sci 36, 625-632, doi:10.1016/j.tibs.2011.08.008S0968-0004(11)00137-X [pii] (2011).
4 Bierhaus, A. et al. Diabetes-associated sustained activation of the transcription factor nuclear factor-kappaB. Diabetes 50, 2792-2808 (2001).
5 Verdier, Y., Zarandi, M. & Penke, B. Amyloid beta-peptide interactions with neuronal and glial cell plasma membrane: binding sites and implications for Alzheimer's disease. J Pept Sci 10, 229-248, doi:10.1002/psc.573 (2004).
Williams, J. H. & Ireland, H. E. Sensing danger—Hsp72 and HMGB1 as candidate signals. J Leukoc Biol 83, 489-492, doi:jlb.0607356 [pii]10.1189/jlb.0607356 (2008).
7 Chiodoni, C., Colombo, M. P. & Sangaletti, S. Matricellular proteins: from homeostasis to inflammation, cancer, and metastasis. Cancer Metastasis Rev 29, 295-307, doi:10.1007/s10555-010-9221-8 (2010).
8 Sevillano, N. et al. Internalization of the receptor for advanced glycation end products (RAGE) is required to mediate intracellular responses. J Biochem 145, 21-30, doi:10.1093/jb/mvn137mvn137 [pii] (2009).
9 Rojas, A. et al. The receptor for advanced glycation end-products: a complex signaling scenario for a promiscuous receptor. Cell Signal 25, 609-614, doi:10.1016/j.cellsig.2012.11.022S0898-6568(12)00325-7 [pii] (2013).
10 Turovskaya, O. et al. RAGE, carboxylated glycans and S100A8/A9 play essential roles in colitis-associated carcinogenesis. Carcinogenesis 29, 2035-2043, doi:10.1093/carcin/bgn188bgn188 [pii] (2008).
11 Gebhardt, C. et al. RAGE signaling sustains inflammation and promotes tumor development. J Exp Med 205, 275-285, doi:10.1084/jem.20070679jem.20070679 [pii] (2008).
12 Taguchi, A. et al. Blockade of RAGE-amphoterin signalling suppresses tumour growth and metastases. Nature 405, 354-360, doi:10.1038/35012626 (2000).
13 Hiwatashi, K. et al. A novel function of the receptor for advanced glycation end-products (RAGE) in association with tumorigenesis and tumor differentiation of HCC. Ann Surg Oncol 15, 923-933, doi:10.1245/s10434-007-9698-8 (2008).
14 Liliensiek, B. et al. Receptor for advanced glycation end products (RAGE) regulates sepsis but not the adaptive immune response. Journal of Clinical Investigation 113, 1641-1650, doi:Doi 10.1172/Jci200418704 (2004).
15 Zhang, L. et al. Receptor for advanced glycation end products is subjected to protein ectodomain shedding by metalloproteinases. J Biol Chem 283, 35507-35516, doi:10.1074/jbc.M806948200M806948200 [pii] (2008).
16 Dykxhoorn, D. M. RNA interference as an anticancer therapy: a patent perspective. Expert Opin Ther Pat 19, 475-491, doi:Doi 10.1517/13543770902838008 (2009).
17 Ramachandran, P. V. & Ignacimuthu, S. RNA Interference as a Plausible Anticancer Therapeutic Tool. Asian Pac J Cancer P 13, 2445-2452, doi:Doi 10.7314/Apjcp.2012.13.6.2445 (2012).
18 Ducry, L. & Stump, B. Antibody-Drug Conjugates: Linking Cytotoxic Payloads to Monoclonal Antibodies. Bioconjugate Chem 21, 5-13, doi:Doi 10.1021/Bc9002019 (2010).
19 Doronina, S. O. et al. Enhanced activity of monomethylauristatin F through monoclonal antibody delivery: effects of linker technology on efficacy and toxicity. Bioconjug Chem 17, 114-124, doi:10.1021/bc0502917 (2006).
20 Graf, I. Remington's Pharmaceutical Sciences, 17th Ed.: 100 Years. Hrsg. von The Philadelphia Coll. of Pharmacy and Science, Editor A. R. Gennaro; Mack Publishing Comp., Easton, Pennsylv. 1985, Vertrieb durch J. Wiley & Sons, Ltd, Chichester, W.-Sussex (Engl.), 1984, S. 21×28, £ 85,50 (netto). Pharmazie in unserer Zeit 14, 191-191, doi:10.1002/pauz.19850140607 (1985).

1. A therapeutic agent comprising a monoclonal antibody that binds the extracellular domain of Receptor for Advanced Glycation End Products (RAGE) linked to an anti-cancer drug.

2. The therapeutic agent of claim 1, wherein the monoclonal antibody specifically binds to SEQ ID NO 24, or to a V-region of Receptor for Advanced Glycation End products (RAGE).

3. The therapeutic agent of claim 1, wherein the monoclonal antibody binds at least a part of the V-region, comprising amino acids 23 to 116 of SEQ ID NO 1.

4. The therapeutic agent of claim 1, wherein the monoclonal antibody is a human or humanised antibody.

5. The therapeutic agent of claim 1, wherein the anti-cancer drug is a cytotoxin; a hormone; a cytokine, chemokine, or other cell signaling molecule; or a nucleic acid.

6. The therapeutic agent of claim 1, wherein the monoclonal antibody is linked to the anti-cancer drug by way of a chemical linking group.

7. The therapeutic agent of claim 6, wherein the linking group is chemically labile, enzymatically labile, or non-cleavable.

8. The therapeutic agent of claim 6, wherein the linking group is a maleimidocaproyl-valine-citrullin-p-aminobenzyloxycarbonyl linker.

9. The therapeutic agent of claim 1, wherein the ratio of drug to antibody is from about 1:1 to 1:8.

10. The therapeutic agent of claim 1, wherein the ratio of drug to antibody is from about 1:1.5 to 1:3.5.

11. A pharmaceutical composition comprising the therapeutic agent of claim 1 and a pharmaceutically acceptable carrier.

12. A method for preparing the therapeutic agent of claim 1, comprising linking together a monoclonal antibody that binds the Receptor for Advanced Glycation Endproducts (RAGE) and an anticancer agent.

13. The method of claim 12, wherein, in a first step, a linking group is added to the anticancer agent, and one or more of the resulting product is reacted with the monoclonal antibody.

14. The method according to claim 12, wherein the ratio of anticancer agent to antibody is from about 1:1 to 1:8.

15. The method according to claim 13, wherein said linking group is chemically labile, enzymatically labile, or non-cleavable.

16. The method according to claim 13, wherein the linking group is a maleimidocaproyl-valine-citrullin-p-aminobenzyloxycarbonyl linker.


SEQUENCE LISTING (28 sequences)

SEQ ID NO: 1 - protein, 404 aa, Homo sapiens
  MISC_FEATURE: Receptor for advanced glycation end products (RAGE)
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 2 - protein, 335 aa, Homo sapiens
  MISC_FEATURE: RAGE variant
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGEGFDKVREAEDSPQHM

SEQ ID NO: 3 - protein, 420 aa, Homo sapiens
  MISC_FEATURE: RAGE
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVVEESRRSRKRPCEQEVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 4 - protein, 390 aa, Homo sapiens
  MISC_FEATURE: RAGE Variant
MAAGTAVGAWVLVLSLWGAVVGAQNITARIGEPLVLKCKGAPKKPPQRLEWKLGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 5 - protein, 363 aa, Homo sapiens
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVVEESRRSRKRPCEQEVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGEGFDKVREAEDSPQHM

SEQ ID NO: 6 - protein, 351 aa, Homo sapiens
  MISC_FEATURE: RAGE variant
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGLRTREPTAVWPPIPATGPRKAVLSASASSNQARRGQLQVRGLIKSGKQKIAPNTCDWGDGQQERNGRPQKTRRKRR

SEQ ID NO: 7 - protein, 347 aa, Homo sapiens
  MISC_FEATURE: RAGE variant
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGEGFDKVREAEDSPQHM

SEQ ID NO: 8 - protein, 342 aa, Homo sapiens
  MISC_FEATURE: Rage variant
MAAGTAVGAWVLVLSLWGAVVGAQNITARIGEPLVLKCKGAPKKPPQRLEWKLGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDVSDLERGAGRTRRGGANCRLCGRIRAGNSSPGPGDPGRPGDSRPAHWGHLVAKAATPRRGEEGPRKPGGRGGACRTESVGGT

SEQ ID NO: 9 - protein, 325 aa, Homo sapiens
  MISC_FEATURE: Rage variant
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDNQARRGQLQVRGLIKSGKQKIAPNTCDWGDGQQERNGRPQKTRRKRRSVQN

SEQ ID NO: 10 - protein, 347 aa, Homo sapiens; identical to SEQ ID NO: 7

SEQ ID NO: 11 - protein, 36 aa, Homo sapiens
  MISC_FEATURE: Rage variant
MAAGTAVGACASGGGPIGGGARRWSSSSWWNRNPDL

SEQ ID NO: 12 - protein, 121 aa, Homo sapiens
  MISC_FEATURE: RAGE variant
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNWWWSQKVEQ

SEQ ID NO: 13 - protein, 153 aa, Homo sapiens
  MISC_FEATURE: Rage variant
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGILGGLGTAALLIGVILWQRRQRRGEERKAPENQEEEEERAELNQSEEPEAGESSTGGP

SEQ ID NO: 14 - protein, 155 aa, Homo sapiens [sequence omitted]

SEQ ID NO: 15 - protein, 260 aa, Homo sapiens
  MISC_FEATURE: Rage variant
MERRPSPTTESVSTSLRTFTITASDWIFPPSEIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGEGFDKVREAEDSPQHM

SEQ ID NO: 16 - protein, 246 aa, Homo sapiens [sequence omitted]

SEQ ID NO: 17 - protein, 260 aa, Homo sapiens [sequence omitted]

SEQ ID NO: 18 - protein, 245 aa, Homo sapiens
  MISC_FEATURE: Rage variant
MGSPWCLMRRGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 19 - protein, 246 aa, Homo sapiens
  MISC_FEATURE: Rage variant
MNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGEGFDKVREAEDSPQHM

SEQ ID NO: 20 - protein, 250 aa, Homo sapiens
  MISC_FEATURE: Rage variant
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWGEHRWGGPQAHVSTFWKSDP

SEQ ID NO: 21 - protein, 121 aa, Homo sapiens; identical to SEQ ID NO: 12
  MISC_FEATURE: Rage variant

SEQ ID NO: 22 - protein, 388 aa, Homo sapiens
  MISC_FEATURE: Rage variant
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRAELNQSEEPEAGESSTGGP

SEQ ID NO: 23 - protein, 149 aa, Homo sapiens
  MISC_FEATURE: Rage variant
MNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWGEHRWGGPQAHVSTFWKSDP

SEQ ID NO: 24 - protein, 28 aa, Homo sapiens
  MISC_FEATURE: Rage variant
  MISC_FEATURE: Rage fragment
SISIIEPGEEGPTAGSVGGSGLGTLALA

SEQ ID NO: 25 - protein, 113 aa, Mus sp.
  MISC_FEATURE: Antibody VH sequence
QVQLQQSGAELVKPGASVKLSCKTSGYTFTNYYIYWVIQRPGHGLEWIGEINPSNGGTNFSERFKSRAKLTVDKPSSTAYMQLSSLTSDDSAVYYCTTNFDYWGQGSTLTVSS

SEQ ID NO: 26 - protein, 112 aa, Mus sp.
  MISC_FEATURE: Antibody VL sequence
DVLMTQTPLSLPVSLGDQASMSCRSSQNIVHNNGNTYLQWYLQKPGQSPKLLIYQVSNRFFGVPDRFSGSGSGTDFTLKISRVEAEDLGVYYCFQGSHLPLTFGAGTKLELK

SEQ ID NO: 27 - protein, 112 aa, Mus sp.
  MISC_FEATURE: Antibody VH sequence
QVQLLQPGAELVRPGASVRLSCKASGYTFTSYWINWVKQRPGQGLEWIGNIYPSDSYTNYNQKFKDKATLTVDKSSSTAYMQLSSPTSEDSAVYYCAREGYWGQGTLVTVSA

SEQ ID NO: 28 - protein, 112 aa, Mus sp.
  MISC_FEATURE: Antibody VL sequence
ELVMTQSPLTLSVTIGQPASISCKSGQSLLYSNGKTYLYWLLQRPGQSPKRLIYLVSKLDSGVPDRFTGSGSGTDFTLKISRVEAEDLGVYYCVQGTHFPYTFGGGTKLEIK